(19) United States
(12) Patent Application Publication
(10) Pub. No.: US 2026/0297144 A1
(43) Pub. Date: Oct. 1, 2026

(54) SIALYLATED HUMAN FACTOR H PROTEIN AND THERAPEUTIC USE THEREOF

(71) Applicant: ELEVA GMBH, Freiburg im Breisgau (DE)

(72) Inventors: Christian SIEVERT, Freiburg im Breisgau (DE); Nicola KRIEGHOFF, Freiburg im Breisgau (DE); Benjamin FODE, Freiburg im Breisgau (DE); Sören BOLLER, Freiburg im Breisgau (DE); Janine ALT, Freiburg im Breisgau (DE); Paulina DABROWSKA-SCHLEPP, Freiburg im Breisgau (DE); Andreas BUSCH, Freiburg im Breisgau (DE); Andreas SCHAAF, Freiburg im Breisgau (DE); Karin HEIDENREICH, Freiburg im Breisgau (DE); Birgit BERG, Freiburg im Breisgau (DE)

(73) Assignee: ELEVA GMBH, Freiburg im Breisgau (DE)

(21) Appl. No.: 19/475,674

(22) PCT Filed: Apr. 29, 2024
(86) PCT No.: PCT/EP2024/061782

§ 371 Date: Oct. 15, 2025

(30) Foreign Application Priority Data
Apr. 28, 2023 (EP) 23170655.7

(51) Int. Cl. C07K 14/47; A61K 38/00; A61P 27/02

(52) U.S. Cl. CPC C07K 14/472; A61P 27/02; A61K 38/00

(57) ABSTRACT

The present invention relates to in vitro sialylated human factor H protein or biologically active sialylated fragments or biologically active sialylated variants thereof. The present invention also relates to in vitro methods of producing such proteins and using such proteins in methods of treating complement-mediated diseases such as C3 glomerulopathy, atypical hemolytic uremic syndrome, or age-related macular degeneration.

The present invention relates to in vitro sialylated human factor H protein or biologically active sialylated fragments or biologically active sialylated variants thereof. The present invention also relates to in vitro methods of producing such proteins and using such proteins in methods of treating complement-mediated diseases such as C3 glomerulopathy (C3G), atypical hemolytic uremic syndrome (aHUS), or age-related macular degeneration (AMD).

The glycoprotein complement factor H (FH) plays a central role in the regulation of the alternative pathway (AP) of complement activation, and mutations as well as antibodies affecting FH function lead to severe renal diseases, like atypical hemolytic uremic syndrome or C3 glomerulopathies. Polymorphisms in FH are also associated with a frequent form of visual impairment, age-related macular degeneration. In aHUS, FH dysfunction leads to uncontrolled complement activation and ultimately, formation of the C5b-9 terminal membrane attack complex on glomerular endothelial cells. This is accompanied by cell damage, glomerular thrombotic microangiopathy, and acute renal failure (ARF), historically resulting in death or terminal renal failure in >60% of genetically determined patients. In C3G, which progresses to terminal renal failure in >50% of patients within 10 years, pathognomonic complement depositions are found in the glomerular basement membrane. Uncontrolled AP activation in C3G is caused by mutations in complement genes, especially FH, or autoantibodies affecting C3 convertase activity.

Therapeutic treatment options for aHUS and C3G are limited and include plasmapheresis or substitution with fresh frozen plasma (FFP) and immunosuppressive treatment as well as renal transplantation, with a high risk of recurrence of the underlying disease. Among currently investigated complement targeting therapeutics, eculizumab, a humanized monoclonal anti-C5 antibody, blocks C5b-9 formation and has been approved for aHUS treatment. However, due to the blocking mode of eculizumab the desired ability of the complement system to inhibit bactericidal activity in the patient is impaired as well. For C3G, no therapeutic regime has been established yet. The application of eculizumab in patients with C3G led to a partial response in only some patients.

FH regulates the activation of C3 convertases in serum as well as on cell surfaces. It consists of 1213 amino acids with a structure of 20 repetitive globular short consensus repeat (SCR) domains, 40 disulphide bonds, and nine putative sites for asparagine (Asn, N)-linked glycans, mainly occupied by nonfucosylated diantennary disialylated glycans on Asn511, −700, −784, −804, −864, −893, −1011, and −1077 of the mature protein, resulting in a molecular mass of 155 kD (for sequence reference see UniProtKB—P08603-1). SCRs 1~4 contain the complement regulatory region of the protein. This region binds to C3b and competes with factor B (FB), thus inhibiting C3-convertase (C3bBb) formation and further promoting the dissociation of the preformed C3 convertase (decay acceleration activity). Additionally, these domains are relevant for FH to act as a cofactor for factor I (FI)-dependent, irreversible cleavage and inactivation of C3b into iC3b. The C-terminus of FH (SCRs 18-20) represents the surface-binding region that interacts with C3b, C3d, and cellular surfaces. Binding of FH on cell surfaces or biologic membranes is mediated by polyanionic structures, like proteoglycans or sialic acids, or glycosaminoglycans (e.g., heparin) and controls local complement activation on endogenous cells, such as glomerular endothelial cells. The majority of FH mutations found in patients with aHUS directly influence the ability of FH to interact with C3b or host cells, thus causing alterations in the activity of this important complement regulator (http://www.fh-hus.org/).

The substitution of FH by administration of FFP can restore normal complement activity in aHUS or C3G, suggesting that FH purified from plasma as well as recombinant FH will be a valuable treatment option for therapy of these patients. In factor H-deficient (FH−/−) mice showing a C3G-like phenotype, the administration of purified plasma-derived human factor H (FHplasma) resulted in the rapid normalization of plasma C3 levels and resolution of the glomerular C3 deposition. Additionally, FHplasma supplementation normalized serum-induced in vitro hemolysis in sera of patients with aHUS.

Biopharmaceutical manufacturing of FH is complex and labour and cost intensive. Recombinant FH has been expressed in mammalian cells, insect cells, yeast and moss. However, these approaches have yet to show therapeutic value and there is still a need in the art for means to provide on an industrial scale recombinant FH which can be used therapeutically to treat FH related diseases. Therefore, it was the object of the present invention to provide recombinant FH which can be used therapeutically.

This problem is solved by the subject-matter as set forth in the appended claims and in the description below.

As will be shown in the following, the inventors of the present invention have surprisingly found that FH protein produced in the moss Physcomitrium patens and subsequently sialylated in vitro can be effectively used as a substitute for serum derived FH protein. FH protein produced in this manner exhibits a glycosylation profile which is not identical to the glycosylation profile of FH protein derived from human serum but surprisingly essentially exhibits nonetheless the same functional properties as serum derived human FH protein. Even more surprisingly, such FH protein exhibits an increased targeting of the kidney as compared to serum derived FH, rendering the recombinant sialylated FH protein particularly suitable for acute treatment of factor H related renal diseases. A further advantage of the moss produced sialylated FH protein is for example, that Neisseria growth is surprisingly inhibited more effectively than with serum derived FH protein or non sialylated moss derived FH protein.

Therefore, the present invention relates in a first aspect to sialylated human factor H protein or a biologically active sialylated fragment or biologically active sialylated variant thereof, wherein the protein, fragment or variant does not comprise trisialylated N-glycans of the structure A3G3S3 (NaNaNa) and/or does comprise monosialylated N-glycans of the structure A1G1S1 (NaM). Also encompassed by this aspect of the invention are pharmaceutical compositions comprising the sialylated human factor H protein or the biologically active sialylated fragment or the biologically active sialylated variant thereof and a pharmaceutically acceptable pharmaceutical acceptable diluent, excipient or carrier.

In a second aspect, the present invention relates to a method of sialylating human FH protein (or fragment or variant thereof), wherein the method comprises the step of in vitro sialylating galactosylated but non-sialylated human FH protein (or fragment or variant thereof).

In a third aspect, the present invention relates to sialylated human factor H (or fragment or variant thereof) produced with a method according to the present invention (second aspect of the invention). Also encompassed by this aspect of the invention are pharmaceutical compositions comprising the sialylated human factor H protein or the fragment or variant thereof and a pharmaceutically acceptable pharmaceutical acceptable diluent, excipient or carrier.

In a fourth aspect, the present invention relates to sialylated human factor H protein according to the first or third aspect of the invention or a pharmaceutical composition comprising the same for use in a method for the treatment of the human body, in particular for the treatment of complement-mediated diseases and disorders such as C3 glomerulopathy, atypical hemolytic uremic syndrome and age-related macular degeneration, in particular dry AMD.

In a fifth aspect, the present invention relates to a method of treating a complement-mediated disease or disorder in a subject, the method comprising administering to the subject a therapeutically effective amount of a factor H protein according to the first or third aspect of the invention or a pharmaceutical composition comprising the same to a subject in need thereof. The complement disease or disorder, without being limited thereto, may for example be selected from the group consisting of C3 glomerulopathy, atypical hemolytic uremic syndrome and age-related macular degeneration, in particular dry AMD.

In a sixth aspect, the present invention relates to a method of galactosylating human FH protein (or fragment or variant thereof), wherein the method comprises the step of in vitro galactosylating non-galactosylated human FH protein. In this context, the present invention also relates to galactosylated but non-sialylated human factor H (or fragment or variant thereof), in particular galactosylated but non-sialylated human FH protein (or fragment or variant thereof) produced with the aforementioned method of galactosylating human FH protein (or fragment or variant thereof).

As mentioned above, the present invention relates to human factor H protein, in particular to sialylated forms thereof. The art knows numerous polymorphisms and sequence variations for human factor H, all of which are encompassed herein by the term “human factor H protein”. For example, database entry UniProtKB—P08603 discloses for human factor H protein numerous natural variants and the present invention is not limited to one particular polymorphism or isoform of human factor H protein. Preferably, the human factor H protein of the present invention is a “mature” factor H protein, i.e. lacks the N-terminal signal peptide. A particularly preferred form of “mature” human factor H according to the present invention comprises the amino acid sequence of SEQ ID NO:1. The term “human factor H protein” relates to the full-length sequence of (mature) human factor H and does not comprise fragments or non-natural sequence variants of factor H protein. Moreover, the term “human factor H protein” defines merely the amino acid sequence of the factor H protein, but does as such not impose any restrictions on post-translational modifications which may or may not be present on the protein. In particular, the term does not require that the factor H protein of the invention exhibits precisely the post translational modifications typically found on factor H protein derived from human serum. The term “sialylated human factor H protein” (or respective sialylated fragment or sialylated variant thereof) requires, that factor H protein exhibits sialylated N-glycans. Preferably, the sialic acid is N-acetyl neuraminic acid (Neu5Ac). Moreover, if herein reference is made to “canonical FH”, the sequence as provided in UniProt Knowledge Database Entry P08603-1 (entry version 245, sequence version 4) including the signal peptide is meant. Any reference to canonical FH herein serves merely illustrative purposes to explain for example the position of certain glycosylation sites. While the specific amino acid sequence of P08603-1 is encompassed by the present invention, the present invention is not limited to specifically this single embodiment of a human factor H amino acid sequence.

The present invention also relates to sialylated fragments and variants of human factor H.

The sialylated FH protein (or a biologically active sialylated fragment or biologically active sialylated variant thereof) of the first aspect of the invention is preferably characterized by not having any trisialylated N-glycans of the structure A3G3S3 (NaNaNa). Generally, the person skilled in the art is readily familiar with the types of N-glycan structures which may be present on glycoproteins and the corresponding nomenclature. In table 1 below typical abbreviations as used herein, Oxford annotation (where possible), sum formulas and exemplary structures for N-glycans relevant for the present invention are provided.

TABLE 1
Summary of relevant N-glycans. Shown are abbreviations, sum formulas
and exemplary structures

| Symbols | | | |
|---|---|---|---|
| 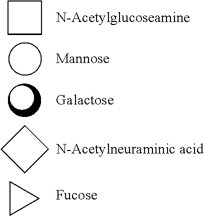 | Hex: Hexose HexNAc: N-Acetylhexosamine Neu5Ac: N-Acetylneuraminic acid meth, Me: methylated dmeth: double methylated dHex: deoxy-Hexose Man: Mannose GlnAc: N-Acetyl-D-glucosamine Gal: galactose Fuc: fucose | | |
| | Oxford | | |
| Abbreviation | Annotation | Sum formula | Exemplary Structures |
| NaM*2Me | | Hex(4)HexNAc(3) Neu5Ac(1)Me(2) or 1x Man+2x Man meth+3x GlnAc+1x Gal+1x Neu5Ac 2x Man+1x Man dmeth+3x GlnAc+1x Gal+1x Neu5Ac | 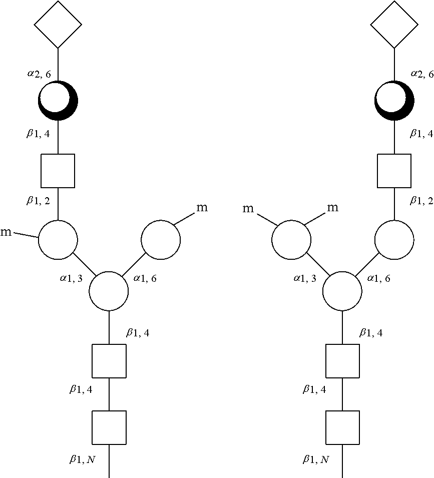 |
| NaM*1Me | | Hex(4)HexNAc(3) Neu5Ac(1)Me(1) or 2x Man+1x Man meth +3x GlnAc+1x Gal+1x Neu5Ac | 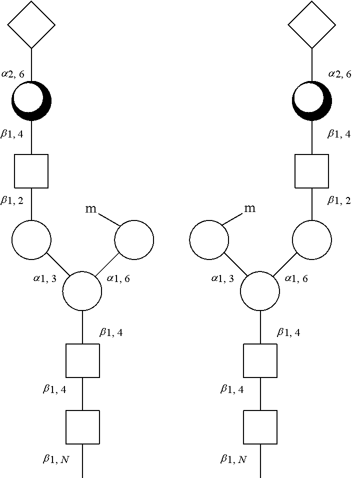 |
| NaM | A1G1S1 | Hex(4)HexNAc(3) Neu5Ac(1) or 3x Man+3x GlnAc+1x Gal+1x Neu5Ac | 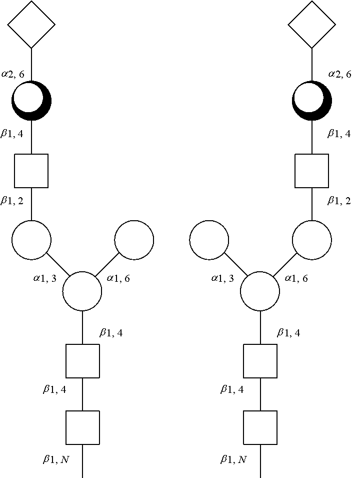 |
| NaA | A2G2S1 | Hex(5)HexNAc(4) Neu5Ac(1) or 3x Man+4x GlnAc+2x Gal+1x Neu5Ac | 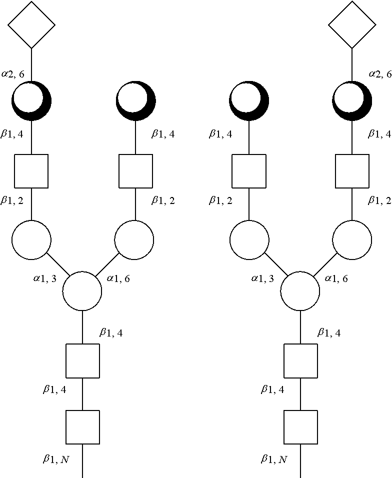 |
| Na(FA) | | Hex(5)HexNAc(4)dHex(1) Neu5Ac(1) or 3x Man+4x GlnAc+2x Gal+1x Neu5Ac+1x Fuc | 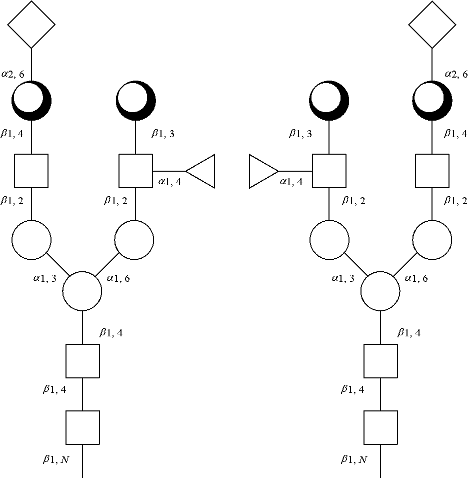 |
| NaNa | A2G2S2 | Hex(5)HexNAc(4) Neu5Ac(2) or 3x Man+4x GlnAc+2x Gal+2x Neu5Ac | 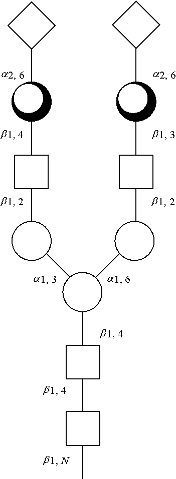 |
| NaNaNa | A3G3S3 | Hex(6)HexNAc(5)Neu5Ac(3) or 3x Man+5x GlnAc+3xGal+3x Neu5Ac | 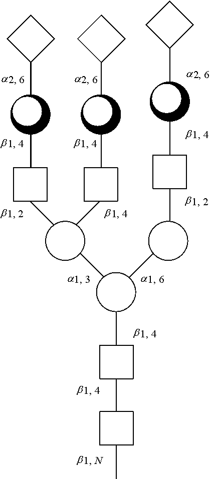 |
| GnM*2Me | | Hex(3)HexNAc(3)Me(2) or 1x Man+2x Man meth+3x GlnAc 2x Man+1x Man dmeth+3x GlnAc | 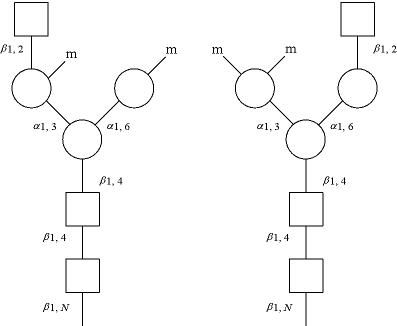 |
| GnM*1Me | | Hex(3)HexNAc(3)Me(1) or 2x Man+1x Man meth+3x GlnAc | 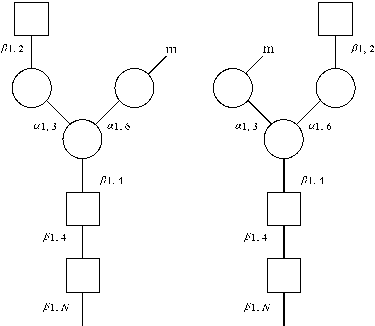 |
| GnGn*1Me | | Hex(3)HexNAc(4)Me(1) or 2x Man+1x Man meth +4x GlnAc | 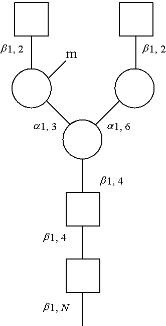 |
| GnM | A1 | Hex(3)HexNAc(3) or 3x Man+3x GlnAc | 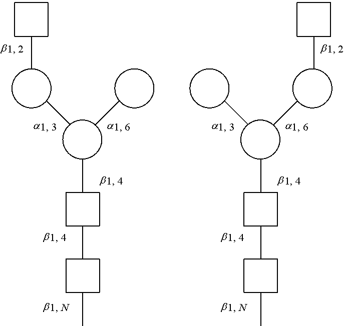 |
| GnGn | A2 | Hex(3)HexNAc(4) or 3x Man+4x GlnAc | 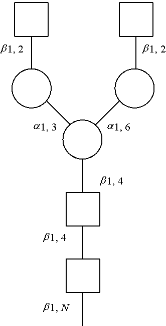 |
| Gn(FA) | | Hex(4)HexNAc(4)dHex(1) or 3x Man+4x GlnAc+1x Gal +1x Fuc | 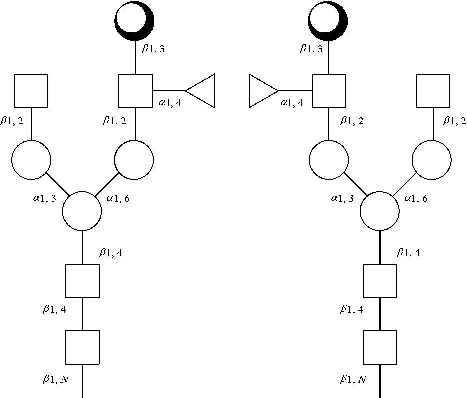 |

Oxford nomenclature uses A for N-acetyl glucosamine, G for galactose, F for fucose, M for mannose, GN for N-acetyl galactosamine and S for a sialic acid, wherein the glycans are read from the backbone to the reducing end and the core made of two GlcNAc and three mannoses, which is common to all N-glycans, is not specifically mentioned.

The inventive sialylated factor H protein (or fragment or variant) according to the first aspect of the invention is preferably characterized by not comprising any trisialylated N-glycans of the structure NaNaNa (A3G3S3 according to the established Oxford annotation; see table 1 above). Trisialylated NaNaNa glycans are typically found on factor H derived from human serum (sd FH), but not on human factor H recombinantly produced initially in the moss Physcomitrium patens and then sialylated in vitro. If herein it is stated that a human factor H protein does not comprise N-glycans of a certain type, e.g. here NaNaNa N-glycans, then this is intended to mean that the respective N-glycan species cannot be detected in a sample of total N-glycans by way of standard means known in the art, i.e. the level is below the detection threshold level of said methods. A corresponding suitable method to determine the presence or absence of N-glycans, such as NaNaNa N-glycans, is provided in the examples section. Human factor H recombinantly produced initially in the moss Physcomitrium and subsequently sialylated in vitro will typically not exhibit any NaNaNa N-glycans and can therefore be easily distinguished from human serum derived FH, which exhibits NaNaNa structures (see for example FIG. 4). Most preferably, the inventive sialylated human factor H protein does not comprise any trisialylated N-glycans at all.

The inventive sialylated human factor H protein (or fragment or variant thereof) according to the present invention may also comprise preferably N-glycan structures of the NaM type. Preferably, less than 20%, even more preferably less than 15% of the total N-glycans of the inventive FH protein (or fragment or variant) are N-glycan structures of the NaM type. “Total N-glycans”, as used herein, is intended to refer to those N-glycans which can be cleaved from glycosylated factor H protein by way of PNGase F treatment. PNGase F is an amidase and works by cleaving between the innermost N-Acetylglucosamine (GlcNAc) and asparagine residues of high mannose, hybrid, and complex oligosaccharides from N-linked glycoproteins and glycopeptides. This results in a deaminated protein or peptide and free glycans. These free glycans (representing the total N-glycans of the glycoprotein) can then be quantitively and qualitatively analyzed in more detail. An exemplary method for determining the total N-glycans of factor H and the presence and absence of different types of N-glycans and their amounts is provided in the examples section. A typical range for NaM N-glycan structures on the sialylated human factor H protein (or fragment or variant thereof) is for example about 5% to about 15% of the total N-glycans. Most preferably, the inventive sialylated human factor H protein (or fragment or variant) does not comprise trisialylated N-glycans of the structure NaNaNa but does comprise monosialylated N-glycans of the structure NaM.

Preferably, the inventive sialylated human factor H protein (or fragment or variant thereof) comprises NaNa N-glycan structures (A2G2S2, see table 1 above). Even more preferably, at least 40%, at least 45%, or even at least 50% of the total N-glycans of the FH protein (or fragment or variant) are NaNa N-glycan structures. Most preferably, the inventive sialylated human factor H protein (or fragment or variant thereof) comprises NaNa N-glycan structures in the range of about 45% to about 60% of the total N-glycans of the FH protein (or fragment or variant). These glycan structures are for example abundantly present in human serum derived FH protein as well as on human factor H recombinantly produced initially in the moss Physcomitrium patens and subsequently sialylated in vitro (see FIG. 4).

Similarly, the inventive sialylated human factor H protein (or fragment or variant thereof) comprises preferably NaA (A2G2S1, see table 1 above) N-glycan structures. Even more preferably, at least about 3.0% of the total N-glycans of the protein (or fragment or variant) are NaA N-glycan structures. For example, about 5% to about 15% of the total N-glycans may be NaA N-glycan structures. Preferably, the total N-glycans of the inventive sialylated human FH protein comprise equal to or less than 15% NaA N-glycan structures.

The N-glycans of the inventive factor H protein may also comprise methylated N-glycans (e.g. NaM*1Me, see table 1). For sake of clarity it is noted that wherever herein reference is made to a given glycan structure without the suffix “*xMe” (wherein x denotes the number of methylations), non-methylated glycan structures are meant. Although no explicit Oxford annotation is available for methylated N-glycans, an NaM*1Me structure is for example a monomethylated A1G1S1 structure, a NaM*2Me structure is a dimethylated A1G1S1 structure, etc. Preferably, less than 20%, even more preferably less than 15% of the total N-glycans of the inventive FH protein (or fragment or variant) are methylated N-glycans, such as NaM*1Me and NaM*2Me. For example, the inventive sialylated human FH protein (or fragment or variant) may comprise less than 10% NaM*1Me N-glycan structures and/or less than 5% NaM*2Me N-glycan structures. A typical range for NaM*1Me N-glycan structures is for example 5 to 10% of the total N-glycans. A typical range for NaM*2Me N-glycan structures on the inventive FH protein (or fragment or variant) is for example 2.0 to 4.0% of the total N-glycans.

The inventive sialylated human factor H protein (or fragment or variant thereof) may carry methylated N-glycan structures, such as the afore-mentioned N-glycan structures of the NaM*1Me type, or of NaM*2Me type. While a methyl group on a sugar residue is seemingly a rarely reported event and is in particular not found in mammals, it is present in some species of bacteria, fungi, algae and plants. For example, human factor H protein produced in the moss Physcomitrium patens and subsequently sialylated in vitro can exhibit such methylation modification.

Another post-translational modification which is not found in humans but can be found is plants are β1,2-Xylose- or α1,3-Fucose-containing N-glycan structures. However, preferably the inventive sialylated human factor H protein (or fragment or variant thereof) does not comprise β1,2-Xylose- or α1,3-Fucose-containing N-glycan structures. This can be achieved even in cases where the inventive human FH protein is produced in plants, such as Physcomitrium patens, for example by knocking out the respective genes for the alpha-1,3-fucosyltransferase (FT3) and beta-1,2-xylosyltransferase (XT), i.e. the enzymes which lead to β1,2-Xylose- or α1,3-Fucose-containing N-glycan structures.

The inventive sialylated human factor H protein (or fragment or variant thereof) may comprise α1,4-fucose-containing N-glycan structures of the Na(FA) type. Again, no explicit Oxford annotation is available but an Na(FA) structure is a fucosylated A2G2S1 N-glycan (see table 1). Preferably, less than 20% of the total N-glycans of the inventive FH protein (or fragment or variant) are α1,4-fucose-containing N-glycan structures of the Na(FA) type. A typical range for Na(FA) N-glycan structures is for example 10 to 15% of the total N-glycans, more preferably 11 to 14%.

In a particularly preferred embodiment the inventive sialylated human factor H protein (or fragment or variant thereof) is characterized by exhibiting one, two or more of the following: NaNa N-glycan structures in the range of about 45% to about 60% of the total N-glycans, NaA glycan structures in the range of about 5% to about 15% of the total N-glycans, Na(FA) glycan structures in the range of about 10% to about 15% of the total N-glycans and NaM glycan structures in the range of about 5% to about 15% of the total N-glycans. Most preferably, the inventive sialylated human factor H protein (or fragment or variant thereof) comprises NaNa N-glycan structures in the range of about 45% to about 60%, NaA glycan structures in the range of about 5% to about 15%, Na(FA) glycan structures in the range of about 10% to about 15% of the total N-glycans and NaM glycan structures in the range of about 5% to about 15%.

Amino acid residues, which are usually glycosylated in human factor H protein and which are preferably also glycosylated in the inventive factor H protein (or fragment or variant thereof) are the asparagine residues at position 529, 718, 802, 822, 882, 911, 1029 and 1095 of canonical FH (see UniProtKB—P08603-1). Importantly, these amino acid positions are provided for sequence P08603-1 of the UniProt knowledge database, which includes the signal peptide of 18 amino acids, and may be at a slightly different position in other human FH proteins, e.g. those lacking the signal peptide. For example, in SEQ ID NO: 1, a preferred amino acid sequence for a factor H protein according to the present invention, the corresponding positions are amino acid residues 511, 700, 784, 804, 864, 893, 1011 and 1077 (due to the lacking signal peptide sequence at the N-terminus). There is another asparagine residue at position 217 (199 in SEQ ID NO:1), which is usually not glycosylated in human FH protein and which is preferably also not glycosylated in an inventive factor H protein.

As mentioned previously, the art discloses multiple polymorphisms for human factor H protein and the present invention is not limited to one particular form of human factor H sequence. However, the inventors of the present invention consider some specific natural variations particularly useful for the purposes of the present invention. For example, it is preferred that the FH protein (or fragment or variant) exhibits at the respective amino acid residue corresponding to position 62 of canonical FH (UniProtKB—P08603-1) valine or isoleucine (corresponds to position 44 in SEQ ID NO:1), with isoleucine being more preferred than valine. Likewise, it is preferred if the FH protein (or fragment or variant) exhibits at the respective amino acid residue corresponding to position 402 of canonical FH (UniProtKB—P08603-1) histidine or tyrosine (corresponds to position 384 in SEQ ID NO:1), with tyrosine being more preferred than histidine. In one embodiment, the FH protein (or fragment or variant thereof) exhibits at the respective amino acid residues corresponding to positions 62 and/or 402 of canonical FH (UniProtKB—P08603-1) one of the following: I62, Y402 or I62 and Y402 (corresponding to I44, Y384 or I44 and Y384 in SEQ ID NO:1). Most preferably, the sialylated human factor H protein according to the present invention comprises the amino acid sequence of SEQ ID NO:1. As mentioned above, the sialylated human factor H protein according to the present invention represents preferably mature FH protein, i.e. does not comprise the FH signal peptide (see amino acids 1 to 18 of canonical FH).

An FH protein according to the present invention is biologically active, i.e. has at least one activity in common with factor H derived from human serum. For example, an FH protein (or fragment or variant thereof) according to the present invention will preferably be capable of binding human C3b protein. A corresponding test is provided in the examples section of the present application. Similarly, an FH protein (or fragment or variant thereof) according to the present invention can preferably proteolytically cleave the α-chain of C3b. A corresponding test is again provided in the examples section of the present application. Likewise, an FH protein (or fragment or variant thereof) is preferably capable to protect sheep erythrocytes from lysis. A corresponding test (hemolysis test) is provided in the examples section of the present application. In this context, the inventors note that to be protected from lysis does not imply that no lysis occurs but that statistically significant less lysis occurs in presence of inventive FH protein than in absence of inventive FH protein. Most preferably, the inventive FH protein protects sheep erythrocytes in the same range (+/−15-20%) as human serum derived FH protein does, e.g. at 100 nM. Furthermore, an FH protein (or fragment or variant thereof) according to the present invention can preferably interact with glycosaminoglycans on cell surfaces. Most preferably, the inventive FH protein has two or more or even all of these properties (binding human C3b protein, proteolytically cleaving of the α-chain of C3b protein, protecting sheep erythrocytes from lysis and interacting with glycosaminoglycans on cell surfaces).

Preferably, a sialylated FH protein (or fragment or variant thereof) of the invention is capable to reduce the number of glomerular C3 deposits in a mouse model of C3G. Glomerular C3 deposition and low serum C3 levels are characteristic pathologic abnormalities in patients with C3G. FH−/− mice, for example, also develop abnormal glomerular C3 accumulation and low serum C3 levels due to AP overactivation, which can be revoked by human FH supplementation, thus providing a useful C3G-model for testing therapeutic efficiency of recombinant FH. A corresponding assay is provided in the examples section of the present application. Most preferably, the inventive FH protein reduces the number of glomerular C3 deposits in the same range as human serum derived FH protein does.

Preferably, an inventive sialylated FH protein according to the present invention is still detectable in the serum of FH−/− mice 24 hours after i.v. injection of 1 mg FH protein into the tail vain of FH−/− mice. A corresponding readout assay to test this requirement is provided in the examples section of the present application. Preferably, the inventive sialylated FH protein has a half time in plasma of mice in the range of 1.5 to 3 hours, preferably of about 2.0 to 3.0 hours, most preferably of about 2.6 to 2.7 hours.

In a second aspect, the present invention relates to an in vitro method of producing sialylated human FH protein (or fragment or variant thereof), wherein the method comprises the step of in vitro sialylating galactosylated but non-sialylated human FH protein. “In vitro”, as used in the context of methods of the present invention, is intended to be limited to ex vivo methods which do not involve living cells. Sialylation processes occurring in a living cell are thus not encompassed by the term “in vitro” as used herein, irrespective whether the cell is a cell in a multi-celled organism (e.g. in a mammal), a cell isolated form a multi-celled organism (e.g. an immortalized cell line) or is itself a single-celled microorganism (e.g. E. coli, yeast). One way to accomplish the sialylation is to contact in vitro (i.e. in a suitable container) galactosylated but non-sialylated human FH protein (or fragment or variant thereof) with sialyltransferase (α-2,6) and CMP-N-acetylneuraminic acid, under conditions allowing sialylation of the galactosylated N-glycans of the FH protein (or fragment or variant thereof).

The human factor H protein (or fragment or derivative thereof) to be sialylated in the method of the second aspect of the invention may be any galactosylated but non-sialylated human FH protein (or fragment or derivative thereof) known to the skilled person. The FH protein (or fragment or derivative thereof) is galactosylated but non-sialylated human FH protein (or fragment or derivative thereof), i.e. it carries N-glycans which are terminally modified with galactose but does not carry N-glycans which are terminally modified with a sialic acid moiety. The method is not limited to a particular polymorphism or naturally or non-naturally occurring variant of human FH protein. However, as above for the FH protein according to the first aspect of the invention, the inventors of the present invention consider some specific natural variations particularly useful to carry out the method of the second aspect of the present invention. In particular, all embodiments set out above for the sialylated human factor H protein (or fragment or derivative thereof) of the first aspect of the invention (and here in particular the mentioned glycan composition and glycan percentages) are likewise suitable embodiments for the human FH protein (or fragment or derivative thereof) to be sialylated with the method of the second aspect of the invention, except that the respective FH protein (or fragment or variant) is of course merely galactosylated and not yet sialylated. For example, as set out above, the FH protein according to the first aspect of the invention may comprise monosialylated N-glycans of the structure A1G1S1 (NaM). A corresponding inventive galactosylated but non-sialylated human FH protein (or fragment or derivative thereof) will thus comprise non-sialylated N-glycans of the structure A1G1. Therefore, all embodiments set out above for sialylated N-glycans apply likewise for the galactosylated but non-sialylated human FH protein except for the presence of the sialic acid moiety, which is necessarily absent. A galactosylated but non-sialylated human FH protein (or fragment or derivative thereof) falls within the scope of the present invention, as discussed below in the context of the sixth aspect of the invention. As before, it is preferred that the galactosylated FH protein (or fragment or variant) exhibits at the respective amino acid residue corresponding to position 62 of canonical FH (UniProtKB—P08603-1) valine or isoleucine (corresponds to position 44 in SEQ ID NO:1), with isoleucine being more preferred than valine. Likewise, it is preferred if the galactosylated FH protein (or fragment or variant) exhibits at the respective amino acid residue corresponding to position 402 of canonical FH (UniProtKB—P08603-1) histidine or tyrosine (corresponds to position 384 in SEQ ID NO:1), with tyrosine being more preferred than histidine. In one embodiment, the galactosylated FH protein (or fragment or variant thereof) exhibits at the respective amino acid residues corresponding to positions 62 and/or 402 of canonical FH (UniProtKB—P08603-1) one of the following: I62, Y402 or I62 and Y402. Most preferably, the galactosylated human factor H protein according to the present invention comprises the amino acid sequence of SEQ ID NO:1. As mentioned above, the galactosylated human factor H protein according to the present invention represents preferably mature FH protein, i.e. does not comprise the FH signal peptide (see amino acids 1 to 18 of canonical FH). Preferably, the galactosylated human FH protein to be sialylated in the method of the second aspect of the invention is galactosylated mature human FH protein. Most preferably, the galactosylated human FH protein to be sialylated in the method comprises the amino acid sequence of SEQ ID NO: 1. Preferably, the asparagine residues corresponding to the positions at 529, 718, 802, 822, 882, 911, 1029 and 1095 of canonical FH (see UniProtKB—P08603-1) are (glycosylated and terminally) galactosylated. Preferably, the asparagine residue at position 217 of canonical FH (199 in SEQ ID NO:1), is not glycosylated (and consequently not galactosylated) in the human FH protein (or fragment or variant) to be sialylated with the method of the second aspect of the invention. The human factor H protein (or fragment or derivative thereof) may have been produced and glycosylated in a plant host cell, preferably in Physcomitrium patens, even more preferably in an alpha-1,3-fucosyltransferase (FT3) and beta-1,2-xylosyltransferase (XT) double knock-out strain of Physcomitrium has then subsequently been galactosylated in vitro (see below).

Sialylation of the galactosylated human factor H protein (or fragment or derivative thereof) can be achieved for example by enzymatical means. In this case the method of the second aspect of the invention comprises the step of contacting in vitro a galactosylated but non-sialylated human FH protein or a fragment or variant thereof with, e.g., sialyltransferase (α-2,6) and CMP-N-acetylneuraminic acid, under conditions allowing sialylating the galactosylated N-glycans of the galactosylated FH protein or of the fragment or of the variant thereof. The sialyltransferase (α-2,6) adds the sialic acid N-acetylneuraminic acid (Neu5Ac) to the terminal galactosylated portions of the N-linked sugar chains of the FH protein (or fragment or derivative thereof). Preferably, the sialyltransferase (α-2,6) will sialylate all galactosylated N-glycans of the FH protein (or fragment or derivative thereof), e.g. the asparagine residues corresponding to the positions at 529, 718, 802, 822, 882, 911, 1029 and 1095 of canonical FH (see UniProtKB—P08603-1). Neu5Ac is generally the preferred sialic acid to be used for sialylation.

In a particularly preferred embodiment of the method of the second aspect of the present invention the method comprises galactosylating in vitro a glycosylated but non-galactosylated and non-sialylated human factor H protein or a fragment or variant thereof in a first step, e.g. by contacting in vitro said glycosylated but non-galactosylated and non-sialylated human factor H protein or a fragment or variant thereof in a first step with an enzyme such as galactosyl transferase (β-1,4) and UDP-galactose, under conditions allowing galactosylation of the N-glycans of the factor H protein, or of the fragment or of the variant thereof, and contacting then in a further subsequent step in vitro the galactosylated human factor H protein or fragment or variant thereof with sialyltransferase (α-2,6) and CMP-N-acetylneuraminic acid, under conditions allowing sialylating the previously galactosylated N-glycans of the factor H protein, or of the fragment or of the variant thereof. For the non-galactosylated and non-sialylated human factor H protein or a fragment or variant thereof the same considerations apply as disclosed above for factor H protein (or fragment or derivative thereof) of the first aspect of the invention. For example, the FH protein according to the first aspect of the invention may comprise monosialylated N-glycans of the structure A1G1S1 (NaM). A corresponding non-galactosylated and non-sialylated human FH protein (or fragment or derivative thereof) will thus comprise non-sialylated non-galactosylated N-glycans of the structure A1. Therefore, all embodiments set out above for galactosylated and sialylated N-glycans apply likewise for the non-galactosylated and non-sialylated human FH protein except for the presence of the galactose and sialic acid moiety, which are necessarily absent. The two-step method of first galactosylating factor H protein, or the fragment or the variant thereof, and in a second step sialylating then the now galactosylated factor H protein, or the fragment or the variant thereof is particularly useful in embodiments where the human factor H protein (or fragment or variant thereof) has been produced and glycosylated in a plant host cell, preferably in Physcomitrium patens, even more preferably in an alpha-1,3-fucosyltransferase (FT3) and beta-1,2-xylosyltransferase (XT) double knock-out strain of Physcomitrium.

As mentioned above, the present invention relates to sialylated human factor H (or fragment or variant thereof) sialylated with a method according to second aspect of the invention. Such compound is herein also encompassed when reference is made to an inventive FH protein or an FH protein according to the present invention. Also encompassed by this aspect of the invention are pharmaceutical compositions comprising the inventive human factor H protein or the fragment or variant thereof sialylated with the inventive method and further comprising a pharmaceutically acceptable diluent, excipient or carrier.

The fourth aspect of the invention relates to an inventive FH protein (or fragment or variant thereof), i.e. a sialylated human FH protein (or fragment or variant thereof) according to the first or third aspect of the invention, or a pharmaceutical composition comprising the same for use in a method for the treatment of the human body. As demonstrated in the examples section, an inventive FH protein (or fragment or variant thereof) is an adequate and fully functional recombinant substitute for human FH protein derived from serum and is for instance capable of binding human C3b protein, proteolytically cleaving the α-chain of C3b and protecting sheep erythrocytes from lysis. It has a reasonable serum half-life and it is capable of reducing the number of glomerular C3 deposits in a mouse model of C3G. The inventive FH protein (or fragment or variant thereof) can therefore be used as a pharmaceutical in factor H related disease states and disorders and can be used to restore normal complement activity. Such disease states and disorders may for example be selected from the group consisting of C3 glomerulopathy, atypical hemolytic uremic syndrome, and age-related macular degeneration, in particular dry AMD. Particularly preferred disease states to be treated with the inventive factor H protein (or fragment or variant thereof) are C3 glomerulopathy, and dry AMD. The method for the treatment of the human body will typically involve administering an effective amount of the inventive FH protein (or fragment or variant thereof). The person skilled in the art will be capable of determining such therapeutically effective amount by routine means. The person skilled in the art will also be capable of determining the most appropriate route of administration for a given disease state or disorder. A particularly preferred clinical dosing range may for example be in the range of 1.5 to 25 mg of an inventive full-length FH protein (or matching amounts of fragments or variants of FH protein corresponding to 1.5 to 25 mg of full-length protein)/kg body weight. Even more preferably, the clinical dosing range is in the range of 3 to 15 mg/kg body weight.

Preferably, the inventive FH protein to be used for treatment of the human body, in particular for the treatment of the aforementioned disease states and disorders, is a sialylated, preferably mature, FH protein comprising SEQ ID NO:1 and exhibiting the glycan composition and percentages as set out above for the first aspect of the invention. Most preferably, such FH protein has been produced in Physcomitrium patens and has then be sialylated with the inventive method according to the second aspect of the invention.

In analogy to the fourth aspect of the invention, the present invention also relates in a fifth aspect of the invention directly to a method of treating a complement-mediated disease or disorder in a subject, the method comprising administering a therapeutically effective amount of a factor H protein according to the first or third aspect of the invention or administering a pharmaceutical composition comprising the same to a subject in need thereof. The complement disease or disorder, without being limited thereto, may for example be selected from the group consisting of C3 glomerulopathy, atypical hemolytic uremic syndrome (aHUS), and age-related macular degeneration (AMD).

Finally, the present invention relates in a sixth aspect to a second method, namely a method of galactosylating human FH protein (or fragment or variant thereof), wherein the method comprises the step of galactosylating in vitro a glycosylated human FH protein (or fragment or derivative thereof). Said glycosylated FH protein is initially glycosylated but non-galactosylated, i.e. it carries initially N-glycans but they do not comprise galactose moieties (let alone sialic acid moieties). The FH protein (or fragment or derivative thereof) can be for example the same as defined for any of the other aspects of the invention, with the proviso that it is not (yet) galactosylated (non-galactosylated) and not (yet) sialylated (non-sialylated). Galactosylation can for example be achieved with an enzyme such as galactosyl transferase (β-1,4) and UDP-galactose. The present invention also relates to galactosylated but non-sialylated human factor H (or fragment or variant thereof), in particular galactosylated human FH protein produced with a method according to the sixth aspect of the invention. Inventive galactosylated but non-sialylated human FH protein (or fragment or variant thereof) may comprise the sequence of SEQ ID NO:1. The galactosylated but non-sialylated human FH protein (or fragment or variant thereof) does preferably not comprise N-glycans of the structure A3G3. Preferably, the galactosylated but non-sialylated human FH protein (or fragment or variant thereof) does comprise N-glycans of the structure A1G1 and/or A2G2. The inventive galactosylated but non-sialylated human FH protein (or fragment or variant thereof) may comprise methylated N-glycans. The inventive galactosylated but non-sialylated human FH protein (or fragment or variant thereof) may be used for example as starting material for the method according to the second aspect of the invention.

The term “comprising”, as used herein, shall not be construed as being limited to the meaning “consisting of” (i.e. excluding the presence of additional other matter). Rather, “comprising” implies that optionally additional matter may be present. The term “comprising” encompasses as particularly envisioned embodiments falling within its scope “consisting of” (i.e. excluding the presence of additional other matter) and “comprising but not consisting of” (i.e. requiring the presence of additional other matter), with the former being more preferred.

### FIGURES

In the following a brief description of the appended figures will be given. The figures are intended to illustrate aspects of the present invention in more detail. However, they are not intended to limit the overall scope of the invention.

FIG. 1 illustrates schematically the complement system and in particular the function of factor H as central regulator of the alternative pathway of the complement pathway (figure taken from Kopp et al, Biomolecules. 2012; 2 (1): 46-75)

FIG. 2A) illustrates the structure of human FH protein (figure taken from Schmidt et al., Protein Expr Purif. 2011; 76 (2): 254-263), including the used glycosylation sites; B) illustrates schematically for one type of N-glycan the two-step sialylation process which can be used to sialylate in vitro FH protein which has been produced in plants such as the moss Physcomitrium patens.

FIG. 3 provides exemplary HILIC-HPLC elution and glycosylation profiles for A) recombinant FH protein produced in the moss P. patents (alpha-1,3-fucosyltransferase (FT3) and beta-1,2-xylosyltransferase (XT) double knock-out), in the following termed “moss-FH”, B) in vitro sialylated recombinant FH protein initially produced in the moss P. patents (FT3 and XT double knock-out), in the following termed “sial-moss-FH”, and for C) human serum derived (sd) FH protein, in the following termed “sd-FH”.

FIG. 4 represents major glycan forms determined via the HILIC-FLD-HPLC analysis of three FH variants: for moss-FH mean results from 9 different production batches, for sial-moss-FH from 4 batches and for sd-FH from one representative batch are presented. “Not assigned, others*” refers to N-glycan structures not already mentioned otherwise in the figure. An obvious difference between the glycan composition of moss-FH and sial-moss-FH is illustrated. In sial-moss-FH the major glycan forms of moss-FH are modified through addition of (Neu5Ac-Gal-GlcNAc) structures. In contrast to sd-FH, sial-moss-FH does not carry the trisialylated NaNaNa glycans.

FIG. 5 provides an in vitro characterization of inventive sialylated FH protein (right) as compared to serum derived FH (left). Specifically, the figure shows in an SDS-PAGE assay with Coomassie staining the dose dependent cleavage of the C3b α-chain, with comparable activities of sd-FH and the inventive sialylated FH protein.

FIG. 6 provides an in vitro characterization of inventive sialylated FH protein in A) an hemolysis assay and B) in a MAC formation assay (TCC ELISA). The inventive FH protein shows the same in vitro activity as serum derived FH. Increasing amounts of inventive sialylated FH protein reduced complement induced hemolysis and MAC (terminal attack complex) formation.

FIG. 7 illustrates that despite shorter half-time in blood, sial-moss-FH shows better/comparable efficacy to sd-FH in a FH (−/−) mouse—an animal model of C3G: A) PK profile of three ¹²⁵I-SIB labelled FH variants in CD-1 mice (n=3); plotted is the plasma FH concentration. Calculated half-times from the early time points (2 min to 6 hours) considering a one-compartment model system: moss-FH: 35 min; sial-moss-FH: 2.66 h; sd-FH: 5.35 h. B) PK profile of three FH variants in FH (−/−) mice; plotted is the serum FH concentration. In correlation to PK profiles obtained in CD-1 mice, moss-FH shows a short half-time with non-detectable levels in serum 4 h post injection. In contrast, sial-moss-FH concentrations are comparable to sd-FH directly post injection, with sd-FH showing slightly longer retention times in serum >24 h post injection. C) sial-moss-FH injected mice show a pronounced increase in serum C3 levels, exceeding the effects achieved using sd-FH; plotted are the serum C3 concentrations. D) Complement C3 kidney deposits 4 days after injection of FH variants into knockout mice, expressed as % of control (PBS-treated) group: C3 deposits in moss-FH injected mice do not differ significantly from PBS injected mice. In contrast, sial-moss-FH injected mice show a pronounced reduction in glomerular C3 deposits, comparable to reduction level achieved in sd-FH treated animals.

FIG. 8 illustrates that sial-moss-FH does not inhibit bactericidal activity of the complement system. Addition of heat inactivated normal human serum (HI NHS) to the test system leads to strong increase of survival of N. meningitidis (with similar levels observed by the inventors after addition of eculizumab to the test system; data not shown). In contrast, addition of sial-moss-FH does only slightly (even less than sd-FH or moss-FH) impact the complement's ability to inactivate N. meningitidis. The tested concentration range represents a doubling of the normal FH concentration (normal FH Concentration 500 mg/L, assay in 50% normal human serum, NHS). 50% NHS were spiked with N. meningitidis and incubated in the presence of different concentrations of sd-FH or sial-moss-FH. Survival of N. meningitidis was scored by counting colony forming units (cfu). Since different serotypes of N. meningitidis are described the most two common serotypes (B and W) were used for experiments. As controls served NHS and heat inactivated NHS. A) serotype W; B) serotype B.

FIG. 9 illustrates the biodistribution of ¹²⁵I-SIB labelled FH proteins (sial-moss-FH and sd-FH) in chosen organs of a CD-1 mouse 30 minutes post intravenous injection. Two different diagrams show better kidney targeting (approx. 2×) of ¹²⁵I-SIB-radiolabelled sial-moss-FH vs. sd-FH: A) % injected dose (% ID) per gram of organ; b) organ to blood concentration ratio: for kidney (sial-moss-FH: 0.59 vs sd-FH: 0.33). Increased targeting of kidney for sial-moss-FH renders the compound a particularly useful candidate for treating complement related renal diseases such as atypical hemolytic uremic syndrome (aHUS) or C3 glomerulopathies (C3Gs).

FIG. 10 illustrates the experimental set up for the assessment the effect of sial-moss-FH (5 μg) on light-induced retinal degeneration compared to PBS vehicle controls in a mouse model of dry-AMD.

FIG. 11 illustrates that sial-moss-FH reduces the extent of degeneration and neuroinflammation, caused by light-induced retinal damage in the eyes of BALB/cJ mice (n=9, since analysis for both eyes, 18 read-outs). A) retinal thickness measured with SD-OCT in circle diameter of 6 mm: sial-moss-FH treated animals show a significantly higher retinal thickness in comparison to PBS treated individuals on day 3 and 4 post light damage. B) TUNEL staining in outer nuclear layer (ONL). TUNEL (Terminal deoxynucleotidyl transferase dUTP nick end labeling) is used to visualize apoptotic cells. Treatment with sial-moss-FH in comparison to PBS markedly reduces the number of apoptotic cells in the ONL following the light damage starting from day 1. C) number of microglia cells (visualized by immunohistochemistry with anti-lba1 antibody) per 15 mm² in subretinal space (SRS). Microglia are the primary immune cells of the central nervous system and are responsible for directing and resolution of immune response. Microglia's translocation to subretinal space is one of the characteristics of the AMD. Pre-treatment with sial-moss-FH significantly reduces the number of microglia cells in SRS vs. PBS treated control. D) Formation of membrane attacking complex (MAC: C5b-9) is markedly reduced after the treatment with sial-moss-FH vs. PBS-treated group starting from day 1. Significance of difference between means was determined by one-way ANOVA and Tukeys multiple comparison test. Data are presented as mean±SEM, *P<0.05, ** P<0.01, *** P≤0.001.

FIG. 12 illustrates that sial-moss-FH treatment reduces the level of proinflammatory cytokine (Il-6) and activation of complement C3 4 days after light-induced retinal damage in the eyes of BALB/cJ mice. (n=9, since analysis for both eyes, 18 read-outs). Transcript levels of: A) C3 in retina measured. B) Il-6 in retina analysed by quantitative real-time PCR. C) Protein levels of Il-6 in retina measured with ELISA. In all parameters significant level reduction has been observed following pretreatment with sial-moss-FH when compared to PBS-treated group. Significance of difference between means was determined by one-way ANOVA and Tukeys multiple comparison test. Data are presented as mean±SEM, *P<0.05, ** P<0.01, *** P≤0.001.

FIG. 13 illustrates experiments carried out for defining a suitable clinical dose range: A) inhibition of hemolysis by addition of increasing amounts of moss-FH with different aHUS patients' sera; Patient 1: C-terminal FH-deletion; Patient 2: FH-Mutation in combination with C3-mutation; Patient 3: FH-mutation R1215Q; Carrier 1: healthy brother of patient 3, carrier of the FH-mutation R1215Q; B) displacement of C3 NEF antibodies by increasing amounts of moss-FH; concentration of MAC was measured by ELISA, plotted are normalized values, 100%=start value of normal human serum (NHS), Patients 1-3 possess C3NEF antibodies.

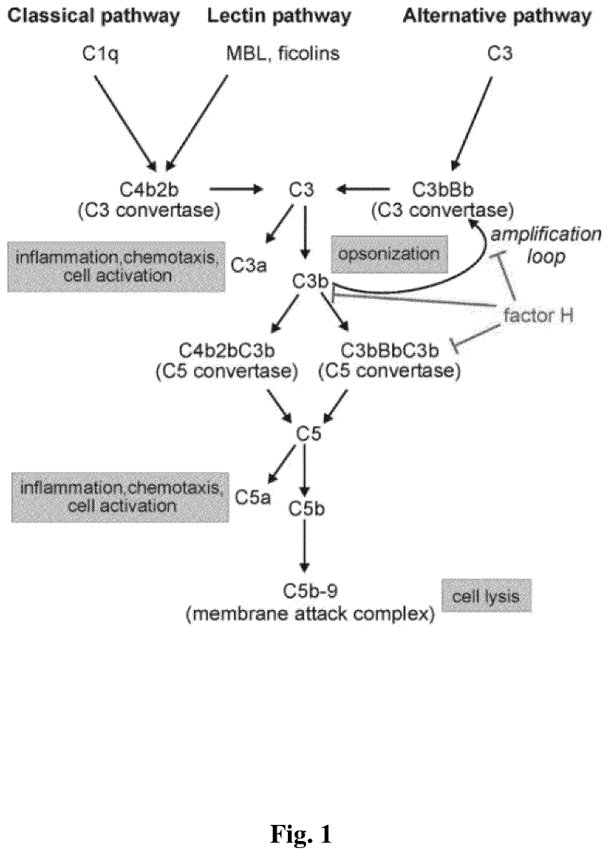

### EXAMPLES

In the following, specific examples illustrating embodiments and aspects of the invention are presented. However, the present invention shall not to be limited in scope by the specific examples described herein. Indeed, various modifications of the invention in addition to those described herein will become readily apparent to those skilled in the art from the foregoing description and the examples below. All such modifications fall within the scope of the appended claims.

### Example 1: Production of Recombinant Factor H Protein in Physcomitrium patens

Recombinant human factor H having the amino acid sequence of SEQ ID NO:1 was produced in P. patents as described previously (Michelfelder et al.; J Am Soc Nephrol.; 2017; 28 (5): 1462-1474). Briefly, the moss Physcomitrium patens can be used for recombinant production of human FH on an industrial scale. However, post-translational modifications have to be considered, especially N-glycosylation, which differs, to some extent, between plants and humans. Although both human and plant glycoproteins harbour diantennary complex-type N-glycans with the identical core structure GnGn (A2 according to Oxford annotation, see table 1), there are two plant-specific sugar residues attached to this core, which are potentially immunogenic in mammals. These sugars, a xylose and a fucose, were completely abolished by genome engineering via homologous recombination, thus modifying the glycosylation pattern of moss for production of human factor H. In the present case, a double knock-out strain of P. patents was used, in which the respective genes for the alpha-1,3-fucosyltransferase (FT3) and beta-1,2-xylosyltransferase (XT) where replaced by knock-out-constructs. Both constructs contain a truncated version of the respective enzyme and additional stop codons. This leads to a premature translation termination and a truncated non-functional enzyme. Hence all proteins in the strains, including the recombinantly produced human FH protein are devoid of the plant specific β1,2-Xylose- or α1,3-Fucose-containing glycan structures. In addition, the recombinant factor H lacks terminal sialic acids on its glycans, which are generally missing in plants.

Production was performed in suspension culture of moss cells in illuminated 500 L single use stirred tank reactors (Sartorius Biostat STR500) followed by purification using standard chromatography and filtration steps.

### Example 2: Sialylation of Recombinant Factor H Protein

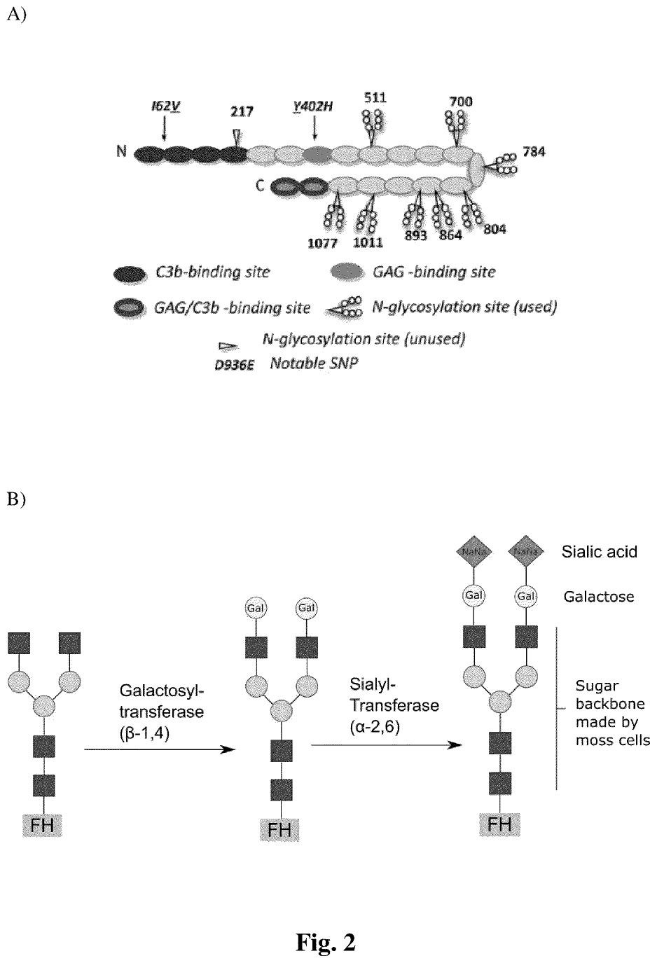

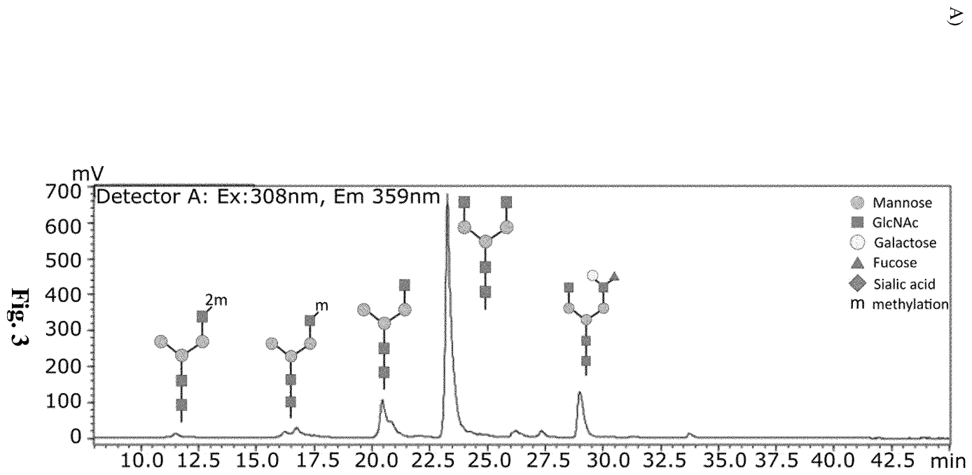

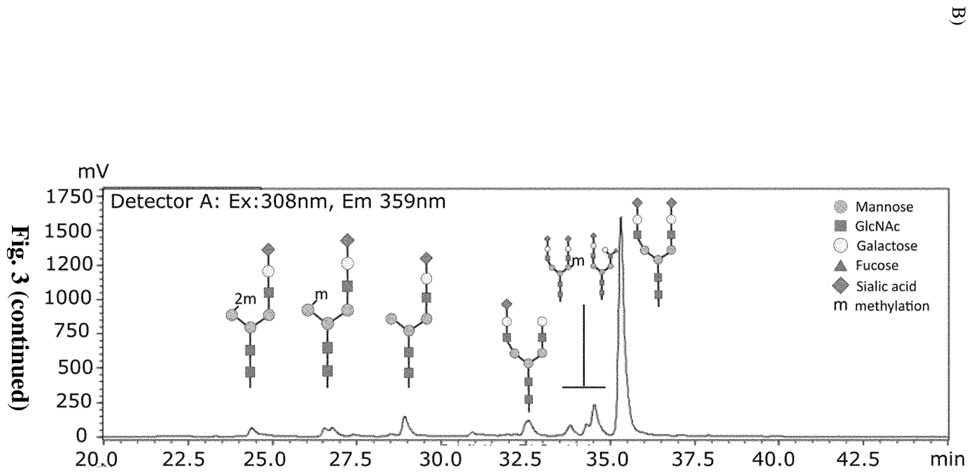

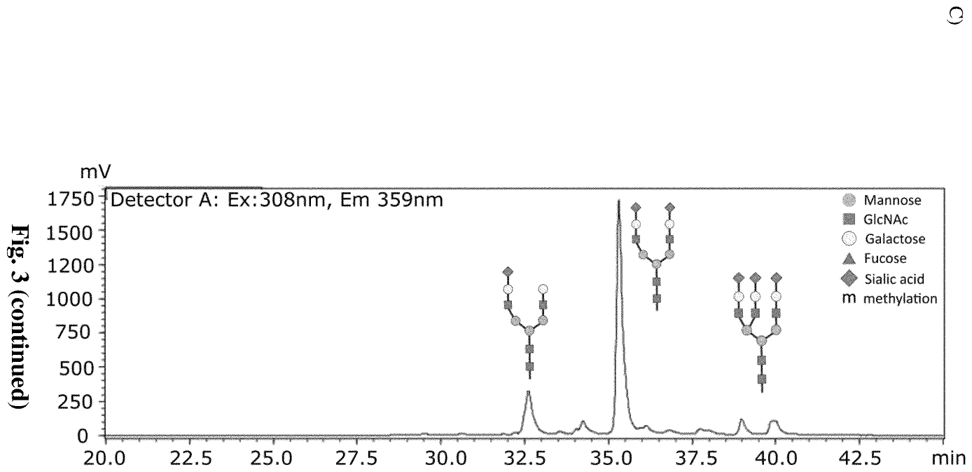

Recombinantly produced FH protein of example 1 was then subjected to a two-step in vitro sialylation as schematically depicted in FIG. 2B. An initial reaction buffer was prepared with conditions adjusted to 80 mM citrate, 4% propylene glycol, 80 mM arginine, 200 mM Tris+0.0025% Tween® 20, pH 6.5. FH concentration was adjusted to 10-15 mg/ml. In a first enzymatic reaction, galactosyltransferase (final 5 mg/L) as well as its substrate UDP-galactose (final 8 mM) and cofactor manganese chloride (MnCl₂) (final 4 mM) were then added. Specifically, substrate and cofactor were added as 10× concentrate. The reaction was then incubated at 37° C. for 7 h. Subsequently, a second enzymatic reaction using sialyltransferase (final 28.5 mg/L) and CMP-N-acetylneuraminic acid (final 2.2 mM) were added, the later as 10× concentrate. At the same time, alkaline phosphatase (final 11 mg/L) and its cofactor zinc chloride (ZnCl₂) (final 50 μM) were added. The phosphatase cleaves CMP, thereby preventing back-reaction of sialyltransferase. This second enzymatic reaction was incubated for 24 h at 37° C.

### Example 3: Structural Analysis of FH Proteins

N-glycan analysis by HILIC-HPLC-MS: To assess identity and quantities of protein linked N-glycans, the glycans have been liberated from the protein and labelled with procainamide. First, 50 μg aliquots were taken in duplicates from the provided glycoprotein solutions and reduced with DTT (final concentration 5 mM) in 100 mM ammonium bicarbonate pH 8.2 at 56° C. for 45 minutes. Iodoacetamide was added to a final concentration of 25 mM and S-alkylation proceeded in the dark for 30 minutes. Samples were then precipitated with 4 times the volume of −20° C. Acetone for 2 hours at −20° C. After washing the pellet with 80%−20° C. Acetone, the samples were shortly dried in vacuo and subjected to PNGase F digest overnight. Liberated N-glycans have been cleaned by passing the reaction solution through a 25 mg C18 Hypersep centrifuge cartridge (Thermo Scientific) and collecting the flow through while retaining proteins. Samples were then dried in vacuo and labelled with procainamide in the presence of cyanoborohydrate (65° C.; 3 h; in dark). After derivatization, a HILIC SPE cleanup was performed on Discovery glycan cartridges (50 mg; Supelco). Bound derivatized glycans were eluted with 500 μL 20% acetonitrile in water. The glycans were dried in vacuo and dissolved in 20 μl water before analysis. Separation was performed on an Acquity UPLC Glycan BEH amide column (2.1×150 mm, 1.7 μm; Waters) with a Security Guard Ultra precolumn (Phenomenex) on a Nexera X2 HPLC system with a RF-20Axs Fluorescence Detector, equipped with a semimicro flow cell (Shimadzu, Korneuburg, Austria). Solvent A consisted of 80 mM formic acid, buffered to pH 4.4 with ammonia, solvent B of 80% acetonitrile in solvent A. The applied gradient started with an initial hold of solvent B at 99% for 8 minutes and a decrease to 57% B over 60 minutes, following 25% B in 2 minutes, at a flow rate of 0.4 ml min-1; the column oven was set to 45° C. and flow cell thermostat to 40° C. Fluorescence was measured with wavelengths Ex/Em 308 nm and 359 nm. Injection volume was 2.5 μl. Peak identity was assessed by coupling the same HPLC system to a Bruker amaZon speed ETD iontrap mass spectrometer equipped with the standard ESI source. Spectra were recorded in the positive ion mode.

### Example 4: Functional Characterization of Sialylated FH Protein In Vitro

### a) C3b Cleavage

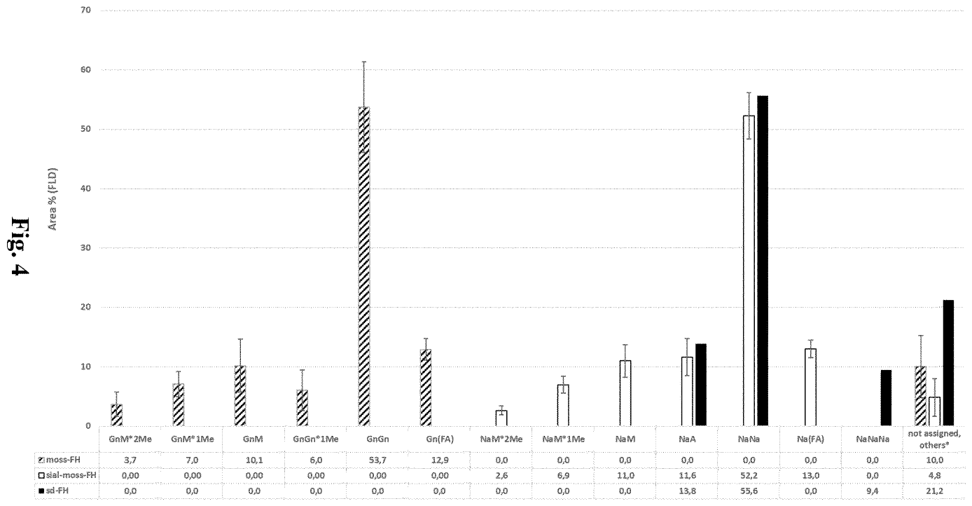

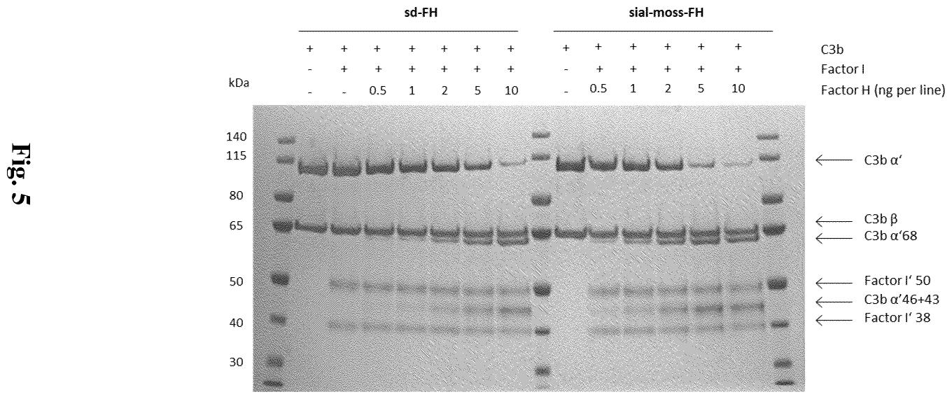

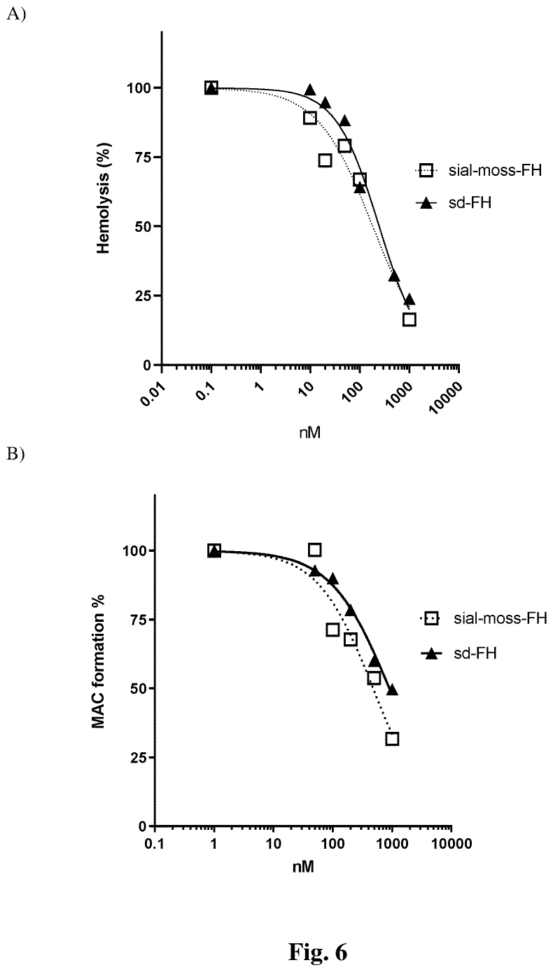

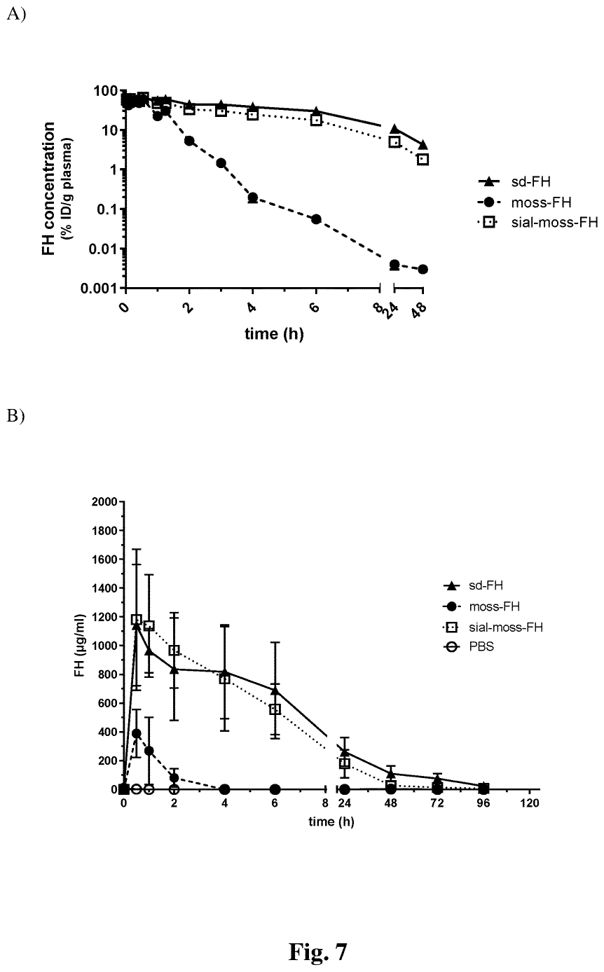

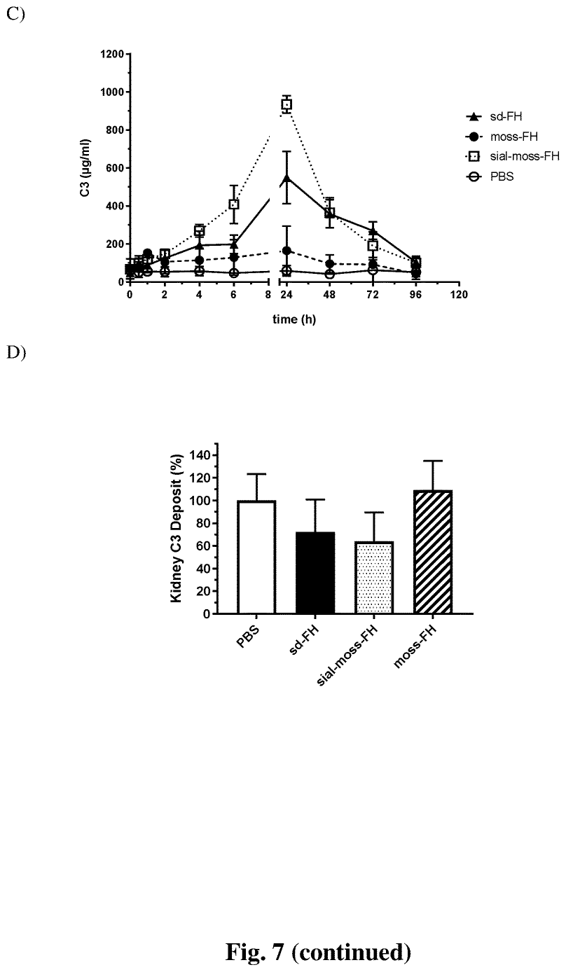

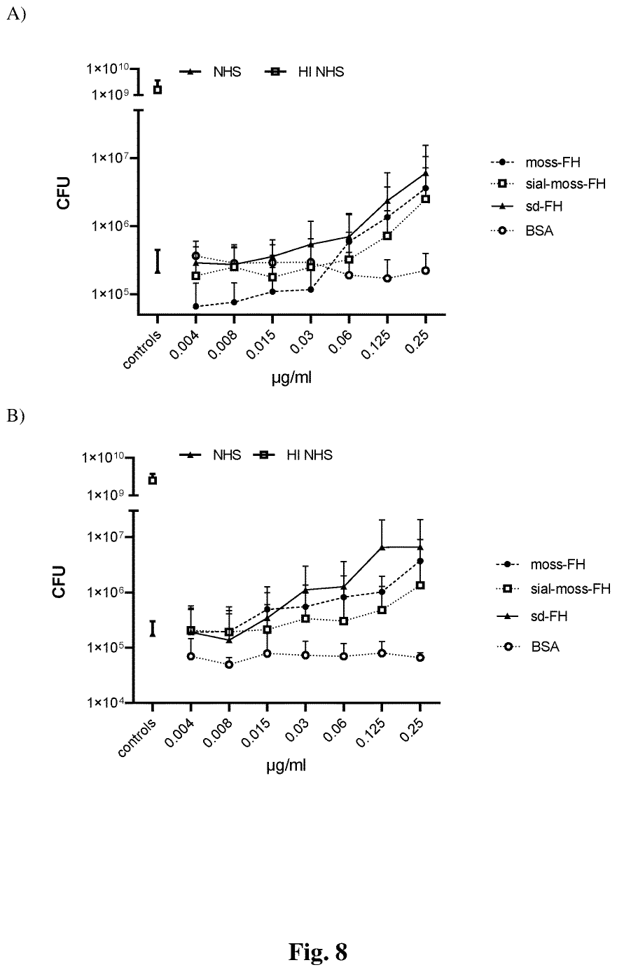

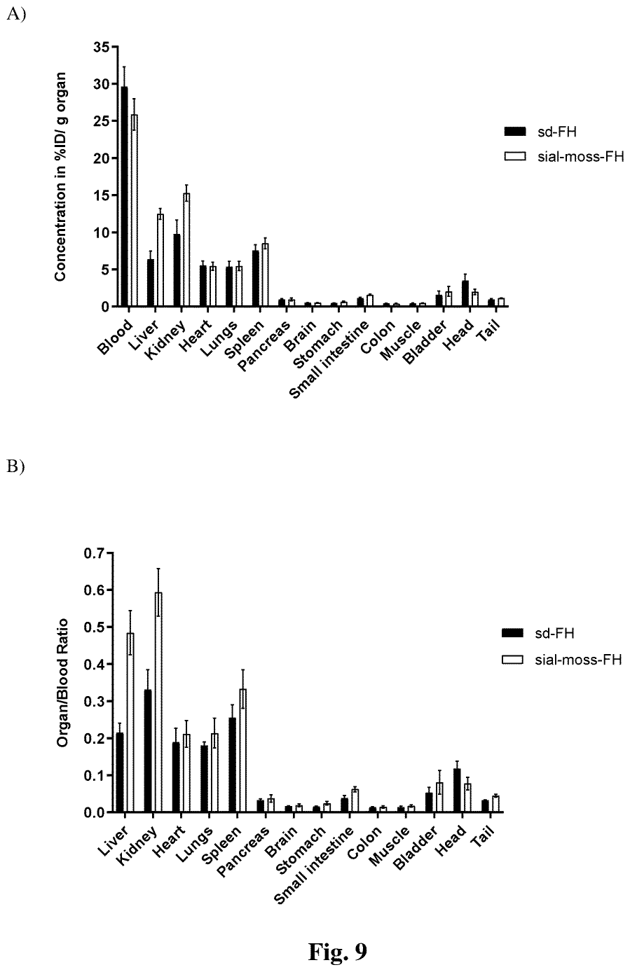

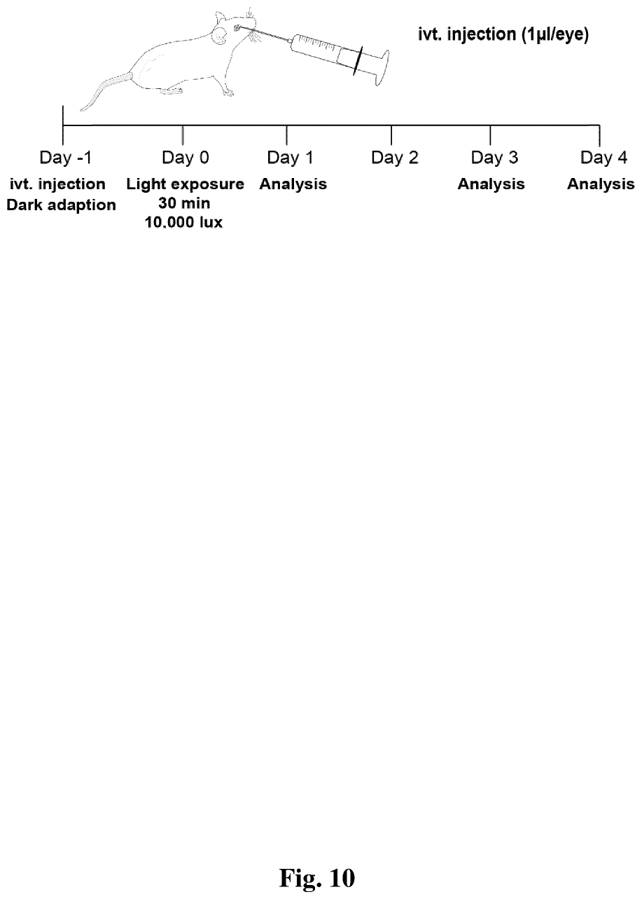

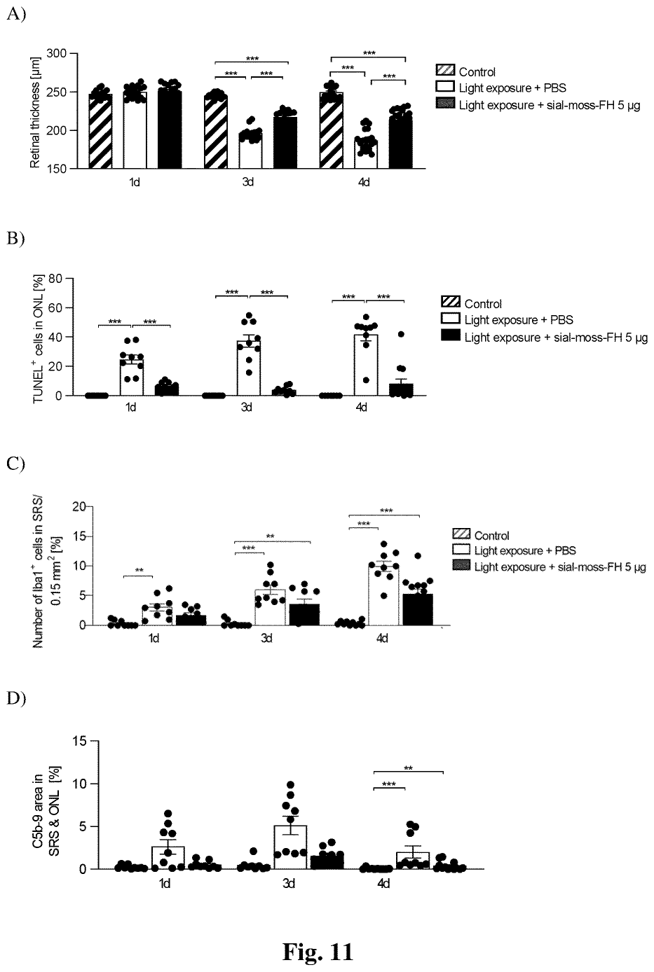

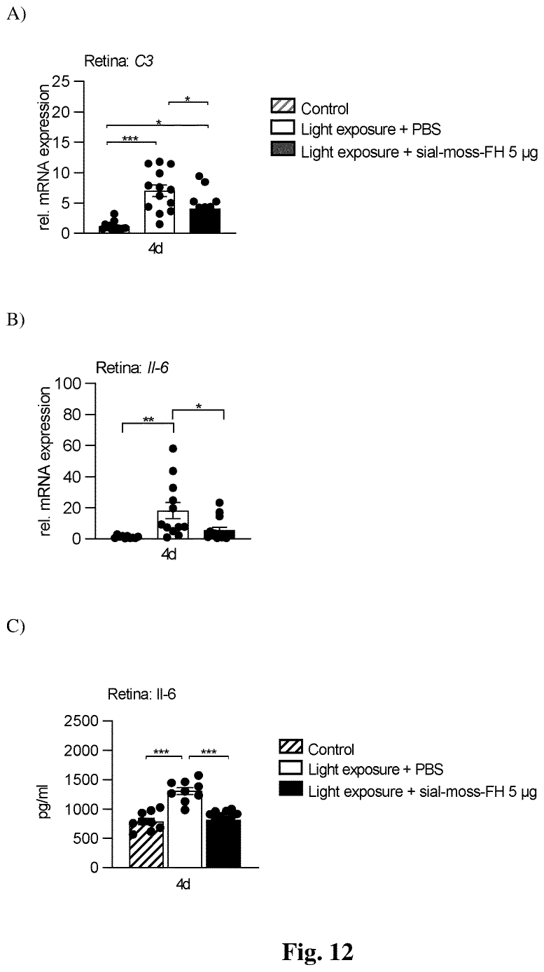

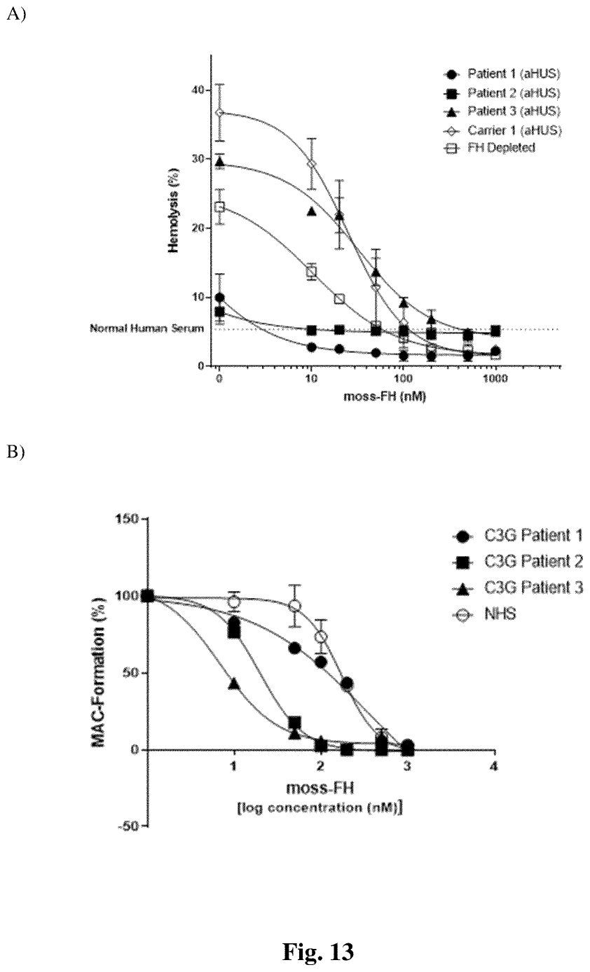

Complement factor H serves as cofactor for complement Factor I whose proteolytic activity cleaves C3b. More precisely, factor I cleaves the α-chain of C3b while the β-chain remains intact. This reaction can be assayed in a simple in vitro assay. Briefly, increasing amounts of sialylated human factor H obtained from example 2 (0.5-10 ng) were mixed with 0.5 μg factor I and 2 μg C3b and incubated at 37° C. for 30 min. Subsequently, degradation of C3b, more precisely degradation of the α-chain of C3b, was visualized via Coomassie stained SDS-PAGE as shown in FIG. 5. C3b consists of an approx. 100 kDa α- and an approx. 65 kDa β-chain which can be separated on a reducing SDS-PAGE. Increasing amounts of sialylated recombinant factor H lead to a dose dependent reduction of intact C3b α-chain and the increase of various degradation fragments with sizes of 43, 46 and 68 kDa.

### b) Protection of Sheep Erythrocytes

Sheep erythrocytes (sE) serve as a model to study alternative pathway (AP) complement activity. Like human erythrocytes, sE express glycosaminoglycans on their surfaces and are normally protected from complement-mediated lysis by plasma-derived FH. Incubation of sE with FH-depleted serum leads to AP-mediated cell surface complement activation, C5b-9 formation, and subsequent hemolysis.

### c) Hemolytic Assay

The hemolytic assay was performed as described before (Sanchez-Corral et al.; Mol Immunol 2004; 41:81-84). Briefly, 5×10⁷ freshly prepared sE were diluted to a final volume of 25 μl in GVB/Mg²⁺/EGTA buffer. Factor H in varying amounts or controls were diluted to a final volume of 15 μl with GVB/Mg²⁺/EGTA buffer and 10 μl of serum was added. Subsequently, the reaction mix was incubated at 37° C. for 30 minutes and stopped by adding 200 μl of GVB/EDTA buffer. After centrifugation, the OD of the supernatants was measured at 414 nm in a microplate reader and corresponding blank values (without serum) were subtracted from each value.

### d) TCC-ELISA, Measurement of MAC Formation

Sial-moss-FH or sd-FH were diluted in NHS serum and incubated in presence of AP-specific buffer in wells pre-coated with LPS. After washing, active C5b-9 formation was detected with alkaline phosphatase-conjugated mAb, recognizing the C9 neoantigen formed during C5b-9 assembly followed by incubation with alkaline phosphatase substrate solution for 30 minutes. Read-out was done at 405 nm in a microplate reader.

### e) C3b Binding

Binding behaviour of sialylated human factor H and sd-FH was analysed using C1 sensor chips in a Biacore T200 instrument for measurements. C3b was first diluted to 0.05 mg/ml in HBS-P buffer (0.01M HEPES pH 7.4, 0.15 NaCl, 0.005% Surfactant P20) and subsequently into a 1 μg/ml solution in acetate buffer (10 mM, pH 5.0). All 4 channels of the chip were first activated with a solution of 60 μl of EDC (N-(3-dimethylaminopropyl)-N′-ethylcarbodiimide hydrochloride) mixed with 60 μl of NHS (N-hydroxysuccinimide). The solution was run at 10 μl/min for 420 seconds until all channels had an RU (response units) of at least 70. A single channel was used as a reference and not immobilized. C3b solution was run through the channels at 5 μl/min until an RU of approx. 150 was reached. After immobilization, all 4 channels were run with ethanolamine solution for 420 seconds at a flow rate of 10 μl/min. Serial dilutions of sd-FH, moss-FH and sial-moss-FH (250, 125, 62.5, 31.3, 15.6, 7.8, 3.9, 2 μM) were generated in HBS-P buffer and ran through the system at 30 μl/min with a 60 second contact time and 300 seconds dissociation time. Analysis temperature and sample compartment temperature was set to 10° C. Kinetic parameters were calculated using Biacore T200 evaluation software.

The table below summarizes the affinity constants that were obtained. The KD ratio between sd-FH and sialylated recombinant FH of example 2 was 2.3-2.8 for all flow channels (Fc with different concentrations of immobilized C3b), showing that the concentration of sd-FH needed for complex formation at equilibrium is approx. 2 times higher than the one needed of FH according to the present invention.

TABLE 2
Affinity constants obtained for sial-moss FH and sd-FH

| Sample | KD (M) | Rmax (RU) | Model | Curve |
|---|---|---|---|---|
| Serum derived FH | 1.10E−07 | 47.3 | Steady State Affinity | Fc = 2-1 |
| Sial-moss FH | 4.82E−08 | 49.6 | Steady State Affinity | Fc = 2-1 |
| Ratio Serum FH/Sial-moss FH | 2.28 | 0.95 | | |
| Serum derived FH | 1.04E−07 | 52.2 | Steady State Affinity | Fc = 3-1 |
| Sial-moss FH | 4.29E−08 | 54.2 | Steady State Affinity | Fc = 3-1 |
| Ratio Serum FH/Sial-moss FH | 2.42 | 0.96 | | |
| Serum derived FH | 1.44E−07 | 36.2 | Steady State Affinity | Fc = 4-1 |
| Sial-moss FH | 5.10E−08 | 34.5 | Steady State Affinity | Fc = 4-1 |
| Ratio Serum FH/Sial-moss FH | 2.82 | 1.05 | | |

### Example 5: Functional Characterization of Sialylated FH Protein in Mice

FH (−/−) knockout mice are characterized by a constantly activated AP which leads to low serum C3 levels and pronounced C3 deposits in the kidney. For the functional characterization of FH protein variant 8-12 weeks old FH (−/−) mice were used (n=3 per time point). Animals were injected intravenously via the tail vain at a dose level of 40 mg/kg corresponding to a volume of 100-150 μL. Control group animals received PBS injection. At determined time points (0; 0.5, 1, 2, 4, 6, 24, 48, 72, 96 h), approximately 60-100 μL of blood was sampled through the tail vain and separated into plasma and serum (blood allowed to clot for 30 minutes and then centrifuged for 10 min at 2000 g). Serum samples were analyzed for FH and C3 concentrations in appropriate ELISA-based assays.

After 96 h (4 days) animals were sacrificed, the kidneys collected into PBS and snap frozen for storage at −80° C. Glomerular C3 deposits were analyzed via fluorescent staining as follows: 10 μm cryosections from mouse kidneys were cut on a Leica CM 3050S cryostat and mounted on SuperFrost® plus glass slides and stored at −80° C. Following fixation in 4% paraformaldehyde solution sections were permeabilized in PBS 0.5% Tween® 20 and C3 was detected using a goat anti-mouse C3 following a second rabbit anti-goat Alexa Fluor 488-conjugated antibody. After washing, slides were mounted in Mounting Medium and covered with glass coverslips. Sections were visualized using a microscope with appropriate software. For quantitative immunofluorescence staining, mean fluorescence intensity of three glomeruli per section was determined by using Image J Software (National Institutes of Health, USA) and expressed in arbitrary fluorescence units (AFU). Change in C3 deposits in each treatment group was then compared (%) to the control (PBS treated) group set to 100%.

### Example 6: Assessment of Inhibition of Neisseria Growth

Sial-moss-FH is expected to support or restore physiological complement regulation by controlling complement overactivation on host cells while retaining protective bacteria-killing properties. To demonstrate this mode of action growth experiments with human serum spiked with Neisseria meningitidis were performed.

N. meningitidis serotype B and N. meningitidis serotype W grown overnight were adjusted to OD 0.1 in 2×THY broth. Subsequently, bacteria were challenged either with pooled complement active human serum (NHS) (50%) or with heat inactivated (1 h 56° C.) human serum (HI-NHS). For bacteria treated with complement active NHS the effect of sial-moss-FH or sd-FH on cell growth was studied. To this end serial dilutions of Factor H were performed in PBS, for concentrations ranging from 0.004, 0.008, 0.015, 0.03, 0.06, 0.125 and 0.25 mg/ml.

As controls Eculizumab and BSA at the same concentrations was added. Bacteria were then incubated for 60 min at 37° C. in presence of 5% CO₂. After serum challenge the samples were put on ice to block complement action. Subsequently, bacteria were serial diluted in THY broth and the diluted samples were transferred to blood agar plates. Following incubation at 37° C. in presence of 5% CO₂ for 24 h the colony forming units (CFU) were counted. Each assay was repeated for each serotype for at least three times, starting from independent cultures.

### Example 7: Biodistribution of ¹²⁵I-SIB Labelled FH Proteins

To evaluate the systemic exposure, pharmacokinetic profile, tissue distribution and the excretion of sial-moss-FH and sd-FH the different FH variants were radiolabeled and administered intravenously to healthy CD-1 mice.

In detail: 1.65 mCi (61 MBq, 0.75 nmol) of iodinated NHS-ester ligand ¹²⁵I-SIB were transferred to a 1.5 mL tube and evaporated to dryness with a stream of nitrogen. To the tube containing ¹²⁵I-SIB, were added 400 μg of FH protein and about 130 μL of 1×PBS pH 7.4. pH solution was adjusted to 8 with addition of 0.2 M borate buffer pH 8.3 to a final volume of 0.2 mL. The mixtures were gently stirred for 30 minutes at room temperature. The volumes were made up to 0.5 mL with 20 mM Tris, 0.15 M NaCl pH 8.5 containing 0.02% PS 20. The percentage of associated radioactivity was determined by instant thin layer chromatography (ITLC) with 10% TCA as eluent. After incubation time, the unreacted ¹²⁵I-SIB was removed by PD-10 desalting column containing Sephadex G-25 resin in with 20 mM Tris, 0.15 M NaCl pH 8.5 containing 0.02% Tween® 20. From the final labeled protein-solution, the radiochemical purity (ITLC/10% TCA precipitation) and the total activity in solution were determined. The quality of radiolabeled proteins has been assessed with radio SE-HPLC and SDS-PAGE. Radio chromatograms of each ¹²⁵I-SIB-factor H variants solutions showed the presence of one major peak (>98%) corresponding to factor H variant as monomer. Electrophoretic profiles obtained after Coomassie blue staining were identical for unlabeled and ¹²⁵I-SIB-factor H variants. Hence, no degradation of the protein through the radiolabeling process in comparison to unlabeled variants could be observed. Dosing solutions were prepared through dilution of labeled sial-moss-FH and sd-FH with each respective unlabeled protein to obtain a specific activity of each dosing solution at about 0.025 mCi/mg (0.925 MBq/mg) and 6.7 mg/mL for FH concentration.

For the in vivo administration: approximately 25-32 g (7 weeks-old) CD-1 mice (n=3 per time point) were used. Anesthetized mice (with isoflurane gas) were injected intravenously with solution at a dose level of 40 mg/kg corresponding to a volume of 148-190 μL and an activity of 0.93-1.19 MBq. This intravenous injection was made via the retro-orbital plexus with a 0.3 mL syringe fitted with a 29-gauge needle. At the intermediate time points, approximately 60-100 μL of blood was sampled from the retro-orbital plexus on anesthetized mice. At the terminal time point, the blood samples were obtained from exsanguination via intracardiac puncture on anesthetized mice by intraperitoneal injection of a mixture of ketamine/xylazine. Blood samples were collected into preweighed Microvette® tubes with heparin lithium (Sarstedt) and the radioactivity was measured in a Gamma counter. Then, blood samples were processed for plasma (centrifugation for 5 min at 2000 g) and plasma samples were also analyzed for their radioactivity in a Gamma counter. The radioactivity in plasma samples was expressed as percentage of the injected dose per gram (% ID/g). For these calculations, the total blood volume of a mouse was estimated as 7.5% of the mouse body weight and its hematocrit value was assumed as 0.53% (Janvier Labs-Hematology data for 10-week old female CD-1 mice). Furthermore, it was assumed a blood density of 1.06 and plasma density of 1. For each compound, the half-life was calculated from the early time points (2 min to 6 hours) considering a one-compartment model system.

At terminal time point (30 min post injection), mice were euthanized by an intraperitoneal overdose of a mixture of ketamine hydrochloride and xylazine hydrochloride and then rapidly sacrificed by exsanguination via intracardiac puncture. Organs of interest were harvested, rinsed in 0.9% NaCl, placed in vials, weighed and radioactivity was measured via gamma counter. The selected tissues/organs were bladder (empty), liver, pancreas, spleen, kidneys, lungs, heart, gastro-intestinal tract (stomach, small intestine, and colon with content), brain, skeletal muscle, thyroid, head and tail. Both kidneys were counted separately. Liver was counted in its entirety by cutting it into two pieces and counting the pieces individually. Concentration of radioactivity was expressed as percentage of the injected dose per gram of organ (% ID/g).

### Example 8: AMD-Experiments

The laboratory for Experimental Immunology of the Eye has established a white light damage paradigm in mouse models to mimic dry-AMD like retinal degeneration can be mimicked in mouse models with established white light damage paradigm (Department of Ophthalmology Experimental Immunology of the Eye at University of Cologne, Germany). In this model, illumination with 10,000 lux white light is used, that contains a blue peak in its spectrum, for 30 min to induce a severe retinal thinning over a time course of four days. The analysis pipeline uses non-invasive in vivo longitudinal spectral domain-optical coherence tomography (SD-OCT) and histology of DAPI-stained retinal cryosections to grade retinal thickness and degeneration. In addition, gene expression and protein analyses together with morphometric analysis of retinal sections and flat mounts documenting microglia number, migration/localization are used to assess the extent of neuroinflammation.

In this mouse model, experiments were performed to assess the effect of sial-moss-FH on light-induced retinal degeneration compared to PBS vehicle controls.

One day before the light exposure induced damage, wild type mice received an intravitreal injection of 5 μg of sial-moss-FH per eye. The control group received a PBS injection. An untreated control group was also included. On Day 0 all animals were exposed to 30 min long light illumination with 10,000 lux white light, that contained a blue peak in its spectrum. On Day 1, 3 and 4 post light exposure, following analyses have been performed and results obtained:

### Measurement of Retinal Thickness

Sial-moss FH treated animals show a significantly higher retinal thickness vs. PBS treated animals on day 3 and 4→rescue of retinal thickness from light induced damage.

### Accumulation of Autofluorescent Material

The degree of measured autofluorescence correlates to the number of “dead” cells. Sial-moss FH treated animals show a significantly lower autofluorescent area vs. PBS treated animals on day 3 and 4→rescue of retinal condition following light damage.

TUNEL (Terminal Deoxynucleotidyl Transferase dUTP Nick End Labeling) is Used to Visualize Apoptotic Cells

Treatment with sial-moss-FH markedly reduced the amount of apoptotic cells following the light damage starting from day 1.

### Measurement of Microglia Reactivity in Subretinal Space (SRS) and in Outer Plexiform Layer (OPL)

Microglia are the primary immune cells of the central nervous system and are responsible for directing and resolution of immune response. Microglia's′ translocation to subretinal space is one of the characteristics of the AMD. Pre-treatment with sial-moss-FH significantly reduces the number of microglia cells in SRS, as well as the number of “dead” microglia cells vs. PBS treated control.

### Formation of Membrane Attacking Complex (MAC:C5b-9):

Treatment with sial-moss-FH markedly reduces the formation of MAC starting from day 1 vs. PBS-treated group

### Müller Cells Gliosis

The major role of the Müller cells is to maintain the structural and functional stability of retinal cells. Indications for potential positive effect of treatment with sial-moss-FH can be observed.

Based on these results it can be concluded that sial-moss-FH significantly reduces the extent of neuroinflammation caused by light-induced retinal damage.

### Example 9: Clinical Dose Range

To establish a clinical dose range for sial-moss-FH, in vitro assays with patient sera of aHUS and C3G patients were used. moss-FH (same amino-acid sequence but differences in glycosylation) was used as surrogate test item for these experiments. The approach is considered valid since it was demonstrated that moss-FH, sial-moss-FH and sd-FH yield a comparable response in the hemolysis assay which is a base of the performed experiments. Hence, calculated dosing range of moss-FH is also considered valid for sial-moss-FH.

Patient sera of aHUS and C3G patients is characterized by the overactive complement system leading to an increased serum C3 depletion and the constant formation of the terminal membrane attack complex (MAC). Since complement factor H regulates complement activity one would expect that an addition of moss-FH to patient's sera leads to a reduction of the complement activity.

Activity of the complement assay was assessed via a sheep erythrocyte hemolysis assay (see above). In this assay sheep erythrocytes are mixed with human serum. Since erythrocytes are susceptible to complement mediated lysis the level of erythrocyte lysis (measured via absorption at 414 nm) is a marker of complement activity level. Normal human serum serves as control (baseline).

The addition of 100-500 nM moss-FH lead to a reduction of complement activity to healthy control level. Based on these results a clinical dosing range of 3-15 mg moss-FH/kg bodyweight was calculated (Table 3).

TABLE 3
Calculation of clinical dosing range;

| | In vitro (20% serum) | | | |
|---|---|---|---|---|
| | 100 nM | | 500 nM | |
| In vivo (patient) | 0.5 | μM | 2.5 | μM |
| | 77.5 | mg/L | 387.5 | mg/L |

| | |
|---|---|
| 3.1 mg/kg body weight* | 15.5 mg/kg body weight* |

*calculation with 40 ml serum/kg body weight

About 80% of C3G patients possess C3NEF antibodies which stabilize the C3 convertase. This in turn leads to an increased C3 cleavage and overactive complement cascade (high amount of terminal membrane attack complex, MAC). Different C3G patients' sera with C3NEF antibodies were supplemented with increasing amounts of moss-FH. Subsequently, the formation of terminal MAC was assessed via ELISA

Addition of increasing amounts of moss-FH led to a reduction of terminal membrane attack complex, indicating that moss-FH can displace C3NEF antibodies and induce dissociation of C3 convertase. This in turn leads to rebalancing of the complement system. Again, a dose between 100-500 nM (equals 3-15 mg moss-FH/kg bodyweight) led to reduction to control level.

On basis of the above data it is concluded that a suitable dosing range for sial-moss FH is preferably in the range of 3 to 15 mg/kg bodyweight.

The following listing of claims replaces all previous listings and versions thereof:

1. Sialylated human factor H (FH) protein, wherein the protein does not comprise trisialylated N-glycans of the structure A3G3S3 (NaNaNa).

2. The sialylated FH protein according to claim 1, wherein at least 40%, preferably at least 45%, even more preferably at least 50% of the total N-glycans of the protein are A2G2S2 (NaNa) N-glycan structures.

3. The sialylated FH protein according to claim 1, wherein at least about 3.0% of the total N-glycans of the protein are A2G2S1 (NaA) N-glycan structures and/or wherein about 5% to about 15% of the total N-glycans of the protein are A1G1S1 (NaM) N-glycan structures and/or wherein about 10% to about 15% of the total N-glycans of the protein are α1,4-fucose-containing N-glycan structures (Na(FA)).

4. The sialylated FH protein according to claim 1, wherein the protein does not comprise (1,2-Xylose- or α1,3-Fucose-containing N-glycan structures.

5. The sialylated FH protein according to claim 1, wherein the respective amino acid residues corresponding to positions 529, 718, 802, 822, 882, 911, 1029 and 1095 of canonical FH (UniProtKB—P08603-1; entry version 245, sequence version 4) are glycosylated in the FH protein, fragment or variant.

6. The sialylated FH protein according to claim 1, wherein the protein exhibits at the respective amino acid residues corresponding to positions 62 and/or 402 of canonical FH (UniProtKB—P08603-1; entry version 245, sequence version 4) one of the following: I62, Y402, or I62 and Y402.

7. The sialylated FH protein according to claim 1, wherein the protein comprises an amino acid sequence according to SEQ ID NO: 1.

8. The sialylated FH protein according to claim 1, wherein the protein binds human C3b protein, proteolytically cleaves the α-chain of C3b and/or protects sheep erythrocytes from lysis.

9. In vitro method of producing sialylated human FH protein, wherein the method comprises the step of in vitro sialylating galactosylated but non-sialylated human FH protein.

10. The method of claim 9, wherein the method comprises the step of contacting in vitro a galactosylated but non-sialylated human FH protein with sialyltransferase (α-2,6) and CMP-N-acetylneuraminic acid, thereby sialylating the N-glycans of the galactosylated FH protein.

11. The method of claim 9, wherein the method comprises the following steps:
a) contacting in vitro a glycosylated but non-galactosylated and non-sialylated human factor H protein with galactosyl transferase (β-1,4) and UDP-galactose, thereby galactosylating the N-glycans of the factor H protein,
b) contacting in vitro the galactosylated human factor H protein with sialyltransferase (α-2,6) and CMP-N-acetylneuraminic acid, thereby sialylating the N-glycans of the galactosylated factor H protein.

12. The method of claim 11, wherein the human factor H protein has been produced and glycosylated in a plant host cell, preferably in Physcomitrium patens, even more preferably in an alpha-1,3-fucosyltransferase (FT3) and beta-1,2-xylosyltransferase (XT) double knock-out strain of Physcomitrium patents.

13. Sialylated factor H protein produced with a method according to claim 9.

14. The sialylated human factor H protein according to claim 1 comprising the same for use in a method for the treatment of the human body.

15. The sialylated human factor H protein or composition for use according to claim 14, wherein the method is a method for the treatment of a complement disease or disorder, such as C3 glomerulopathy, atypical hemolytic uremic syndrome, or age-related macular degeneration, in particular dry AMD.


SEQUENCE LISTING (1 sequence)

SEQ ID NO: 1 - protein, 1213 aa, Homo sapiens
  VARIANT 44: note = corresponds to position 62 of UniProtKB database sequence P08603-1 but with V62I
  VARIANT 384: note = corresponds to position 402 of UniProtKB database sequence P08603-1
  CARBOHYD 511: note = N-linked (GlcNAc...)
  CARBOHYD 700: note = N-linked (GlcNAc...)
  CARBOHYD 784: note = N-linked (GlcNAc...)
  CARBOHYD 804: note = N-linked (GlcNAc...)
  CARBOHYD 864: note = N-linked (GlcNAc...)
  CARBOHYD 893: note = N-linked (GlcNAc...)
  CARBOHYD 1011: note = N-linked (GlcNAc...)
  CARBOHYD 1077: note = N-linked (GlcNAc...)
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR